(19) United States
(12) Patent Application Publication
(10) Pub. No.: US 2026/0215773 A1
(43) Pub. Date: Jul. 30, 2026

(54) ARC-AND-LOOP SUTURE GRASPER FOR GRASPING A SUTURE

(71) Applicant: Applied Medical Technology, Inc., Brecksville, OH (US)

(72) Inventors: Grant Wesley PHILLIPS, Richfield, OH (US); Steven Alfred SOEDER, North Royalton, OH (US); Adam Wesley METCALF, Peninsula, OH (US); George J. PICHA, Brecksville, OH (US)

(21) Appl. No.: 19/476,073

(22) PCT Filed: Feb. 19, 2025
(86) PCT No.: PCT/US2025/016418

§ 371 Date: Oct. 16, 2025

Related U.S. Application Data
(60) Provisional application No. 63/555,329, filed on Feb. 19, 2024.

(51) Int. Cl. A61B 17/04; A61B 17/00

(52) U.S. Cl. CPC A61B 17/0485; A61B 17/0469; A61B 2017/00429

(57) ABSTRACT

An arc-and-loop suture grasper for grasping a suture is provided. The suture grasper comprises a needle, a retriever body, an arced grasper wire, and a looped grasper wire. Translation of the retriever body within a lumen of the needle in a first direction causes the arced grasper wire to move from a first position to a second position, thereby allowing the arced grasper wire to arc away from the grasper wire loop, exposing it, and allowing a suture to fit between the arced grasper wire and the grasper wire loop beyond the lumen. Translation of the retriever body within the lumen in a second direction causes the arced grasper wire to move from the second position to the first position, thereby causing the arced grasper wire to arc toward the grasper wire loop, sequestering it, and grasping the suture between the arced grasper wire and the grasper wire loop.

### REFERENCE TO RELATED APPLICATIONS

This application claims the benefit of U.S. Provisional Application No. 63/555,329, filed on Feb. 19, 2024, which is hereby incorporated by reference.

### FIELD OF THE INVENTION

The invention relates to an arc-and-loop suture grasper for grasping a suture, and more particularly to an arc-and-loop suture grasper for grasping a suture comprising a suture retrieval needle, a retriever body, an arced grasper wire, and a looped grasper wire.

### BACKGROUND OF THE INVENTION

Suture passing is required in many surgical procedures. A suture passer is a surgical instrument which provides a means of delivery and/or retrieval of a suture through some bodily tissue. There are many existing instruments on the market which utilize a mechanical solution to secure a suture for passing.

Increasingly, minimally invasive techniques are being employed over open surgery due to reduction in risk, faster recovery time, and generally better cosmesis. Minimally invasive techniques typically make use of a scope and specialized tools that can be inserted through existing openings of a patient (e.g., via endoscope, colonoscope, etc.) or artificially created openings of the patient (e.g., via laparoscope, arthroscope, etc.) to gain access to the targeted intracorporeal working space.

Working with indirect visualization of surgical instruments through a scope presents a significant technical challenge to the use of suture passers for retrieval. Retrieving a suture using a typical suture passer requires precise positioning and careful manipulation of the suture passer to position one or more grasping elements of the suture passer around the suture. The suture passer must be held relatively steady in position while the grasping elements are closed about the suture, capturing the suture and holding it firmly so it can be retrieved. Skillful manipulation in this manner is hampered by the fact that most scopes employ a single camera and present a two-dimensional image to the suture passer operator and thus do not provide stereoscopic imaging. The lack of stereoscopic imaging hampers the operator's ability to perceive depth, which increases the level of difficulty associated with precisely positioning the grasping elements around the suture. While three-dimensional imaging systems exist, they are expensive and to date remain relatively rare in the field.

Most existing suture passers utilize a multi-arm design, where a grasper end effector including two or more arms are opened and brought around a suture, then closed around the suture to capture it. The arms may be separate, providing a pincer-type grasper with jaws to grasp a suture, or they may be connected, providing a snare-type grasper forming an eye of a loop or hook through which a suture can be threaded. Both designs typically include a hollow hypodermic tube from which the grasping mechanism is extended, allowing the end effector of the grasping mechanism to open. The end effector can then be positioned to encompass the suture. The grasping mechanism is then retracted into the tube, pulling the suture with it. The end effector typically has a width, when open, that is greater than the diameter of the lumen of the tube, so that when the grasping mechanism is retracted, the wall of the tube forces the end effector to collapse upon itself. The grasping mechanism takes up free-space inside the lumen of the tube, blocking the suture from exiting. This passive mechanical pinching of the end effector prevents the release of the suture. The grasping mechanism also can be re-extended from the tube, allowing it to open again and release the suture.

In the pincer-type suture passers, the hypodermic tube is typically ground to a fine point to allow the instrument to pierce skin and soft tissue without the need for a separate needle or instrument. There are some pincer-type suture passers where the grasper end effector does not retract into the lumen of a tube. Instead, the end effector forms a sharp needle tip in its normally closed state. This end effector is basically a set of articulating jaws that perform the grasping operation.

In snare-type suture passers, a loop or a hook is extended from, and retracted into, a hypodermic needle. The surgeon has to feed and/or manipulate the suture through the extended loop or hook before retracting the suture into the hypodermic needle.

Unfortunately, both pincer-type and snare-type suture passers have drawbacks.

Regarding pincer-type suture passers, their end effectors generally have shafts that are relatively large, for example 15 gauge (1.8 mm) or larger. This is a requirement for designs including retractable jaws. This is because such an end effector must be large enough to apply a significant amount of closing force to retain the suture and prevent the suture from slipping. Since the end effector must be relatively large, the corresponding tube also must be relatively large to house the end effector in the retracted state. A large end-effector shaft size also is a requirement for solid needle tip designs. This is because the mechanism to open and close the articulating jaws in these designs must traverse the entire axial length of the device. If the mechanism is not of sufficiently robust size and strength for the suture passer, then the articulating jaws will not have the closing force necessary to retain the suture during use.

Pincer-type suture passers also are unable to allow the administration of contrast, medication, and/or fluid at the tip. Instead, this must be completed by using a separate instrument, through a separate port within the surgical field. In addition, most pincer-type suture passers must be provided with a straight tube having a straight tip at its end. Many surgeons prefer to use surgical tools having a curved tip at the end if possible. This is because a curved tip allows surgeons to have more range of motion at the tip, helping them navigate around features within the surgical field. Suture passers with straight tubes having straight tips make internal manipulation of the suture passer and suture during surgery much more difficult.

Regarding snare-type suture passers, these instruments generally provide a surgical target area that is relatively small. Snare-type suture passers require the surgeon to basically “thread the eye of a needle” within the surgical field. The end of the suture must pass through the small loop/hook at the distal end of the snare-type suture passers. Since laparoscopic cameras used in surgeries only provide a two-dimensional view, the cameras do not provide depth perception for the surgeon. Trying to thread a suture through the small loop or hook without any sense of field depth can be difficult. Moreover, snare-type suture passers also require capturing a large amount of the suture to avoid slippage. This can be problematic for snare-type suture passers using a loop, as these instruments can only effectively grasp a suture at either of its two loose ends, not along the length of the suture. In addition, although snare-type suture passers can have smaller end-effector shafts than pincer-type suture passers, since the snares are only loops of thin wire the holding/retention force of snare-type suture passers can be very weak depending on the size of the suture selected. This can result in slippage of a grasped suture from a snare-type suture passer, even when a large amount of the suture has been captured.

A magnetic U-stitch suturing device intended to address some of these difficulties has been disclosed in Applied Medical Technology, Inc.'s U.S. Pat. No. 10,245,021. The magnetic U-stitch suturing device is made of two hypodermic needles allowing one or more sutures, at the same time, and a retrieval probe to be advanced into a cavity, such as a stomach cavity, of a patient. The one or more sutures can be magnetic sutures, each including a suture magnet, as described in Applied Medical Technology, Inc.'s U.S. Pat. No. 11,672,530. Both the suture and retrieval probe comprise magnets of opposite polarities on their leading ends. Thus, after the suture and retrieval probe are inside the stomach cavity, the suture and retrieval probe may mate and the suture may be transferred from one hypodermic needle to the other using magnetic attraction. In doing so, the suture forms a loop through the stomach. Once removed, this loop, having two ends that are positioned outside the patient's body, can be pulled tight in order to pull the stomach wall closer to the surface of the patient's body. With the stomach wall close to the surface of the patient's body, it is easier to insert a gastrostomy device.

Unfortunately, certain procedures, such as inguinal hernia repair through high ligation of the patent processus vaginalis, require passing of a suture in a space, e.g., a peritoneal cavity, that is not sufficiently large to permit advancement of the two hypodermic needles of the magnetic U-stitch suturing device simultaneously.

Other suture instruments and/or sutures including magnets also have been disclosed. For example, U.S. Pat. No. 10,299,786 discloses a suture insertion device utilizing small gauge needles for threading one or more sutures through subcutaneous tissue. The suture insertion device can include a magnetic capture mechanism for contacting a magnetically attractive strand in transverse alignment. U.S. Pat. Nos. 6,719,765 and 9,770,238 disclose instruments for passing a medical implement through tissue with magnetic forces. U.S. Pat. No. 6,551,304 discloses an apparatus and method for retrieving a remotely located device equipped with a magnetic coupler. U.S. Pat. No. 8,702,753 and U.S. Pub. No. 2008/0243148 disclose sutures to which magnetic anchors are attached. U.S. Pub. No. 2020/0360017 discloses a suturing apparatus in which a suture thread may be automatically passed between a needle and a transfer tube. The suturing apparatus can include electromagnetic coils to engage and release a suture from the system. U.S. Pat. No. 11,490,887 discloses a suturing system including a forceps arm and a suture that may be magnetic to thus engage with each other. U.S. Pub. No. 2022/0104802 discloses a suturing system including a rod having a magnetic tube extending from an end thereof and a magnetic needle having an end attracted into the tube to magnetically engage therewith. U.S. Pat. No. 11,672,530 discloses a magnetic suture that has a ferrule with a tapered region in which a knotted suture is provided and secured with an adhesive and a straight region in which a magnet is provided.

Applied Medical Technology, Inc.'s U.S. Pat. No. 11,839,373 describes a magnet-assisted suture grasper including a suture retrieval needle, a retriever body, a grasper wire, a grasper arm, and a grasper magnet. Translation of the retriever body within a needle lumen of the suture retrieval needle in a first direction causes the grasper arm to move from a first position to a second position, thereby exposing the grasper magnet from the needle lumen and allowing contact between the grasper magnet and a magnetic suture attracted thereto. Translation of the retriever body within the needle lumen in a second direction opposite the first direction causes the grasper arm to move from the second position to the first position, thereby sequestering the grasper magnet and grasping the magnetic suture within the needle lumen.

Applied Medical Technology, Inc.'s U.S. Pat. No. 11,723,655 describes a magnet-assisted suture grasper including a suture retrieval needle, a retriever body, a grasper arm, and a grasper magnet. Another magnet-assisted suture grasper including a handle, a stem, first and second grasper jaws, a grasper magnet, and an actuator body also is described.

Applied Medical Technology, Inc.'s international application PCT/US2022/050444, filed Nov. 18, 2022, describes a magnet-assisted suture grasper for grasping a magnetic suture comprising a suture retrieval needle, a retriever tube, a grasper ferrule, a grasper member, a grasper magnet, and a magnet wire.

Applied Medical Technology, Inc.'s international application PCT/US2022/050475, filed Nov. 18, 2022, describes a magnet-assisted suture grasper comprising a suture retrieval needle, a retriever body, a grasper member, a grasper magnet, and a spring.

Although these magnet-assisted suture graspers provide many advantages, their use is limited in two ways. First, these magnet-assisted suture graspers need to be used with magnetic sutures including a suture magnet. Accordingly, their use requires specialized sutures. Second, these magnet-assisted suture graspers can only be used to grasp a magnetic suture at the end of the magnetic suture at which the suture magnet is attached. This limits the ways that surgeons can grasp and manipulate sutures during surgical procedures.

Considering suture passer designs utilizing a multi-arm design further, U.S. Pat. No. 8,808,313 discloses a surgical instrument having a suture passer/advancer and a suture retriever manipulated from a common handle. The retriever is preferably a loop that closes around the suture advanced through it by the advancer. The loop is retracted to draw it around the suture. The passer/advancer has a fork-like distal end to advance and let go of the suture after it is in the loop of the retriever. Unfortunately, capturing a suture with this suture passer would likely be challenging in the absence of stereoscopic imaging, as it would require engaging the suture in the fork-like distal end of the advancer for delivery of the suture to the retriever.

U.S. Pub. No. 2021/0085311 discloses a suture passer for use in arthroscopic soft tissue repair procedures. The suture passer includes a cannulated needle having a distal tip. A post is slidable within the cannulated needle. A snare is slidable within the cannulated needle between an extended position and a retracted position. In the extended position, the snare extends from the distal tip of the cannulated needle. The snare has a shape such that when the snare is in the extended position, fully extended out from the distal tip of the cannulated needle, the snare bends away from the post to create an open region. In the retracted position, the snare extends around the post. The suture passer may have a handle extending proximally from the cannulated needle. In use, a suture is passed through an open region between the snare and the post in the extended position. As the snare and, in some cases, the post, are retracted, the snare extends around the post and closes the open region, trapping the suture between the snare and the post in a closed region. Unfortunately, operation of this suture passer would appear to require a physician to manipulate the positions of the post and snare independently multiple times during passing of each suture, making the operation complicated.

WO2023/076367 discloses a system for a medical device that can operate multiple actuated implements with sophisticated and individual motion profiles using a single user actuator. The medical device includes a curved piercing cannulated needle that houses two implements, a hook and a lasso, that extend from a tip of the needle to cooperate and entrap a suture. In order to capture the suture, the hook and the lasso are moved independently of each other and through different motion profiles. Unfortunately, this suture passer requires a complicated mechanical system for operation.

Improved suture passers that reduce the technical difficulty associated with capturing and retrieving sutures under indirect non-stereoscopic visualization and in other contexts are needed.

### BRIEF SUMMARY OF THE INVENTION

An arc-and-loop suture grasper for grasping a suture is disclosed. The arc-and-loop suture grasper comprises: (a) a suture retrieval needle comprising a proximal end, a distal end, and a needle body extending therebetween, the needle body defining a needle body axis between the proximal and distal ends of the suture retrieval needle, the needle body having a proximal hole, a distal hole, and a needle lumen extending therebetween along the needle body axis; (b) a retriever body disposed within the needle lumen and translatable therein along the needle body axis; (c) an arced grasper wire having a proximal end and a distal end, the proximal end of the arced grasper wire being fixedly disposed within the retriever body, the arced grasper wire extending distally from the retriever body and being reversibly moveable between a first position and a second position; and (d) a looped grasper wire comprising a proximal portion and a distal portion, the proximal portion of the looped grasper wire being fixedly disposed within the retriever body, the distal portion of the looped grasper wire forming a grasper wire loop, the grasper wire loop having a distal-most loop point, the looped grasper wire extending distally from the retriever body, the arc-and-loop suture grasper sequestering the grasper wire loop within the needle lumen when the arced grasper wire is in the first position and exposing the grasper wire loop from the needle lumen when the arced grasper wire is in the second position. The arced grasper wire and the looped grasper wire are more flexible than the needle body. When the arced grasper wire is in the first position the arced grasper wire extends through the grasper wire loop and the distal end of the arced grasper wire extends further distally than the distal-most loop point of the grasper wire loop. When the arced grasper wire is in the second position the arced grasper wire arcs away from the grasper wire loop and the distal-most loop point of the grasper wire loop extends further distally than the distal end of the arced grasper wire. The arced grasper wire is sufficiently short to exit the grasper wire loop as the arced grasper wire is moved from the first position to the second position and the looped grasper wire translates in unison with the arced grasper wire, and to enter the grasper wire loop as the arced grasper wire is moved from the second position to the first position and the looped grasper wire translates in unison with the arced grasper wire. Translation of the retriever body within the needle lumen in a first direction along the needle body axis causes the arced grasper wire to move from the first position to the second position, thereby allowing the arced grasper wire to exit the grasper wire loop and arc away from the grasper wire loop, exposing the grasper wire loop, and allowing a suture to fit between the arced grasper wire and the grasper wire loop beyond the needle lumen. Translation of the retriever body within the needle lumen in a second direction opposite the first direction along the needle body axis causes the arced grasper wire to move from the second position to the first position, thereby causing the arced grasper wire to arc toward the grasper wire loop and enter the grasper wire loop, sequestering the grasper wire loop, and grasping the suture between the arced grasper wire and the grasper wire loop within the needle lumen.

In some embodiments, the suture retrieval needle is a hypodermic needle.

In some embodiments, the suture retrieval needle is straight, the needle body axis thereby being straight. Also in some embodiments, the suture retrieval needle is curved, the needle body axis thereby being curved.

In some embodiments, the suture retrieval needle has a sharp tip.

In some embodiments, the retriever body comprises a retriever tube having a proximal hole, a distal hole, and a retriever tube lumen extending therebetween, the retriever tube being partially disposed within the needle lumen and translatable therein along the needle body axis, the proximal hole of the retriever tube being in fluid communication with the distal hole of the needle body through the retriever tube lumen and the needle lumen.

In some of these embodiments, the retriever tube comprises stainless steel. Also in some of these embodiments, the retriever tube lumen is the only lumen of the retriever tube. Also in some of these embodiments, translation of the retriever tube within the needle lumen is limited to a straight path as the translation of the retriever tube causes the arced grasper wire to move between the first and second positions. Also in some of these embodiments, the arc-and-loop suture grasper further comprises a grasper ferrule comprising a proximal end, a distal end, and a ferrule body extending therebetween, the ferrule body having a proximal hole, a distal hole, and a ferrule lumen extending therebetween, the grasper ferrule being fixedly disposed within the retriever tube, wherein the arced grasper wire and the looped grasper wire extend distally from the grasper ferrule. In some examples of these embodiments, the grasper ferrule has been crimped onto the arced grasper wire and the looped grasper wire with a crimped portion of the arced grasper wire and a crimped portion of the looped grasper wire being disposed within the ferrule lumen of the grasper ferrule; and the retriever tube has been crimped onto the grasper ferrule, with the grasper ferrule, the crimped portion of the arced grasper wire, and the crimped portion of the looped grasper wire being disposed within the retriever tube lumen. Also in some examples of these embodiments, the retriever tube has been crimped onto the grasper ferrule, the crimped portion of the arced grasper wire, and the crimped portion of the looped grasper wire, with the grasper ferrule, the crimped portion of the arced grasper wire, and the crimped portion of the looped grasper wire being disposed within the retriever tube lumen, and with the crimped portion of the arced grasper wire and the crimped portion of the looped grasper wire being disposed adjacent to the grasper ferrule and not within the ferrule lumen of the grasper ferrule.

In some embodiments, the arced grasper wire is formed from one or more of a shape memory alloy, titanium, stainless steel, or a monofilament polymer.

In some embodiments, the looped grasper wire is formed from one or more of a shape memory alloy, titanium, stainless steel, or a monofilament polymer.

In some embodiments, the arced grasper wire comprises a distal portion that forms an arcuate curve that defines an arcuate-curve interior between the grasper wire and the grasper wire loop when the arced grasper wire is in the second position. In some embodiments, the arced grasper wire comprises a distal portion that forms an arcuate curve that defines an arcuate-curve interior not between the grasper wire and the grasper wire loop when the arced grasper wire is in the second position.

In some embodiments, the arced grasper wire comprises a distal-most portion that extends transversely from a distal portion of the arced grasper wire when the arced grasper wire is in the second position.

In some embodiments, the arced grasper wire and the looped grasper wire each have a diameter, and the diameter of the looped grasper wire is smaller than the diameter of the arced grasper wire. In some of these embodiments, the arced grasper wire has a diameter of 0.0016 inches (0.040 mm) to 0.016 inches (0.40 mm), and the looped grasper wire has a diameter of 0.001 inches (0.025 mm) to 0.010 inches (0.25 mm); the arced grasper wire has a diameter of 0.008 inches (0.20 mm) to 0.012 inches (0.30 mm), and the looped grasper wire has a diameter of 0.003 inches (0.076 mm) to 0.007 inches (0.18 mm); the arced grasper wire has a diameter of 0.006 inches (0.15 mm) to 0.009 inches (0.23 mm), and the looped grasper wire has a diameter of 0.003 inches (0.076 mm) to 0.005 inches (0.13 mm); and/or the arced grasper wire has a diameter of 0.0045 inches (0.11 mm) to 0.006 inches (0.15 mm), and the looped grasper wire has a diameter of 0.0025 inches (0.064 mm) to 0.004 inches (0.10 mm).

In some embodiments, the arc-and-loop suture grasper further comprises a proximal hub attached to the retriever body and extending proximally therefrom. In some of these embodiments, the arc-and-loop suture grasper further comprises a handle attached to the retriever body. In some of these embodiments, the arc-and-loop suture grasper further comprises a return mechanism configured to cause the arced grasper wire to translate from the second position to the first position by translating the retriever body proximally from within the suture retrieval needle, wherein the return mechanism comprises a spring. In some of these embodiments, the spring has a spring constant, and the spring constant can be used to maintain the arced grasper wire in the first position. In some of these embodiments, the arc-and-loop suture grasper further comprises a spring hat, a stay collar, and an end cap wherein: the spring hat is a low-friction component that preloads the spring, maintaining tension, even when the arced grasper wire is in the first position, and that determines available travel of the retriever body, the proximal hub, the arced grasper wire, and the looped grasper wire during use; the stay collar is fixedly attached to the retriever body and prevents the retriever body, the arced grasper wire, the looped grasper wire, and the spring hat from over-traveling; and the end cap can be used to capture the retriever body, the spring hat, the spring, and the stay collar within the handle.

A system for passing a magnetic suture also is disclosed. The system comprises the arc-and-loop suture grasper and a suture.

### BRIEF DESCRIPTION OF THE DRAWINGS

These and other features, aspects, and advantages of the present disclosure are better understood when the following detailed description is read with reference to the accompanying drawings, which are as follows.

FIG. 1 is a perspective view of an embodiment of an arc-and-loop suture grasper for grasping a suture comprising a suture retrieval needle, a retriever body, an arced grasper wire, and a looped grasper wire as disclosed herein, in which the arced grasper wire is in the first position, and the arc-and-loop suture grasper is sequestering the grasper wire loop within the needle lumen.

FIG. 2 is a side view of the arc-and-loop suture grasper of FIG. 1.

FIG. 3 is a top view of the arc-and-loop suture grasper of FIG. 1.

FIG. 4 is a bottom view of the arc-and-loop suture grasper of FIG. 1.

FIG. 5 is a front view of the arc-and-loop suture grasper of FIG. 1.

FIG. 6 is a back view of the arc-and-loop suture grasper of FIG. 1.

FIG. 7 is a side sectional view of the arc-and-loop suture grasper of FIG. 1, viewed along the cutting plane shown in FIG. 5.

FIG. 8 is an expanded side sectional view of the arc-and-loop suture grasper of FIG. 7.

FIG. 9 is an expanded perspective sectional view of the arc-and-loop suture grasper of FIG. 1, in which the suture retrieval needle is partially transparent as shown.

FIG. 10 is a perspective view of the arc-and-loop suture grasper of FIG. 1, in which the arced grasper wire is in the second position, and the arc-and-loop suture grasper is exposing the grasper wire loop from the needle lumen.

FIG. 11 is a side view of the arc-and-loop suture grasper of FIG. 10.

FIG. 12 is a top view of the arc-and-loop suture grasper of FIG. 10.

FIG. 13 is a bottom view of the arc-and-loop suture grasper of FIG. 10.

FIG. 14 is a front view of the arc-and-loop suture grasper of FIG. 10.

FIG. 15 is a back view of the arc-and-loop suture grasper of FIG. 10.

FIG. 16 is a side sectional view of the arc-and-loop suture grasper of FIG. 10, viewed along the cutting plane shown in FIG. 14.

FIG. 17 is an expanded side sectional view of the arc-and-loop suture grasper of FIG. 16.

FIG. 18 is an expanded perspective view of the arc-and-loop suture grasper of FIG. 10.

FIG. 19 is a side view of the suture retrieval needle of the arc-and-loop suture grasper of FIG. 1.

FIG. 20 is a front view of the suture retrieval needle of FIG. 19.

FIG. 21 is a back view of the suture retrieval needle of FIG. 19.

FIG. 22 is a side view of the retriever body of the arc-and-loop suture grasper of FIG. 1.

FIG. 23 is a front view of the retriever body of FIG. 22.

FIG. 24 is a back view of the retriever body of FIG. 22.

FIG. 25 is a side view of the arced grasper wire of the arc-and-loop suture grasper of FIG. 1.

FIG. 26 is a top view of the looped grasper wire of the arc-and-loop suture grasper of FIG. 1.

FIG. 27 is a side view of a grasper ferrule of the arc-and-loop suture grasper of FIG. 1.

FIG. 28 is a front view of the grasper ferrule of FIG. 27.

FIG. 29 is a back view of the grasper ferrule of FIG. 27.

FIG. 30 is a perspective view of an arced grasper wire, a looped grasper wire, and a grasper ferrule of the arc-and-loop suture grasper of FIG. 1, in which the proximal end of the arced grasper wire and the proximal portion of the looped grasper wire have been inserted through the ferrule lumen of the grasper ferrule.

FIG. 31 is a perspective view of an arced grasper wire, a looped grasper wire, and a grasper ferrule of the arc-and-loop suture grasper of FIG. 1, in which the arced grasper wire and the looped grasper wire are positioned adjacent to the grasper ferrule.

FIG. 32 is a side view of a retriever body, an arced grasper wire, a looped grasper wire, and a grasper ferrule of the arc-and-loop suture grasper of FIG. 1, in which the retriever body has been crimped about the arced grasper wire, the looped grasper wire, and the grasper ferrule at the grasper ferrule.

FIG. 33 is a perspective view of a proximal hub of the arc-and-loop suture grasper of FIG. 1.

FIG. 34 is a front view of the proximal hub of FIG. 33.

FIG. 35 is a back view of the proximal hub of FIG. 33.

FIG. 36 is a side view of a spring of the arc-and-loop suture grasper of FIG. 1.

FIG. 37 is a bottom view of a proximal hub, a spring, a retriever body, a suture retrieval needle, an arced grasper wire, and a looped grasper wire of the arc-and-loop suture grasper of FIG. 1, with the arced grasper wire being in the first position and thus the looped grasper wire being sequestered within the needle lumen.

FIG. 38 is a top view of a proximal hub, a spring, a retriever body, a suture retrieval needle, an arced grasper wire, and a looped grasper wire of the arc-and-loop suture grasper of FIG. 1, with the arced grasper wire being in the second position and thus the looped grasper wire being exposed from the needle lumen.

FIG. 39 is a perspective view of the arc-and-loop suture grasper of FIG. 1, in which the arced grasper wire is in the first position, and the arc-and-loop suture grasper is sequestering the grasper wire loop within the needle lumen.

FIG. 40 is an expanded perspective sectional view of the arc-and-loop suture grasper of FIG. 39, in which the suture retrieval needle is partially transparent as shown.

FIG. 41 is a perspective view of the arc-and-loop suture grasper of FIG. 1, in which the arced grasper wire is in a position intermediate between the first position and the second position, allowing the arced grasper wire to arc away from the grasper wire loop and exposing the grasper wire loop.

FIG. 42 is an expanded perspective sectional view of the arc-and-loop suture grasper of FIG. 41, in which the suture retrieval needle is partially transparent as shown.

FIG. 43 is a perspective view of the arc-and-loop suture grasper of FIG. 1, in which the arced grasper wire is in the second position, allowing a suture (not shown) to fit between the arced grasper wire and the grasper wire loop beyond the needle lumen.

FIG. 44 is an expanded perspective view of the arc-and-loop suture grasper of FIG. 43, in which the suture retrieval needle is partially transparent as shown.

FIG. 45 is a perspective view of the arc-and-loop suture grasper of FIG. 1, in which the arced grasper wire is in the second position, and a suture is disposed between the arced grasper wire and the grasper wire loop.

FIG. 46 is an expanded perspective view of the arc-and-loop suture grasper and the suture of FIG. 45.

FIG. 47 is a perspective view of the arc-and-loop suture grasper of FIG. 1, in which the arced grasper wire is in a position intermediate between the second position and the first position, and a suture is disposed between the arced grasper wire and the grasper wire loop.

FIG. 48 is an expanded perspective view of the arc-and-loop suture grasper of FIG. 47, in which the suture retrieval needle is partially transparent as shown.

FIG. 49 is a perspective view of the arc-and-loop suture grasper of FIG. 1, in which the arced grasper wire is in the first position, the arced grasper wire has arced toward the grasper wire loop, the grasper wire loop is sequestered, and the suture is being grasped between the arced grasper wire and the grasper wire loop within the needle lumen.

FIG. 50 is an expanded perspective sectional view of the arc-and-loop suture grasper of FIG. 49, in which the suture retrieval needle is partially transparent as shown.

FIG. 51 is a perspective view of another embodiment of an arc-and-loop suture grasper for grasping a suture comprising a suture retrieval needle, a retriever body, an arced grasper wire, and a looped grasper wire as disclosed herein, in which the arced grasper wire is in the first position, and the arc-and-loop suture grasper is sequestering the grasper wire loop within the needle lumen.

FIG. 52 is a side view of the arc-and-loop suture grasper of FIG. 51.

FIG. 53 is a top view of the arc-and-loop suture grasper of FIG. 51.

FIG. 54 is a bottom view of the arc-and-loop suture grasper of FIG. 51.

FIG. 55 is a front view of the arc-and-loop suture grasper of FIG. 51.

FIG. 56 is a back view of the arc-and-loop suture grasper of FIG. 51.

FIG. 57 is a side sectional view of the arc-and-loop suture grasper of FIG. 51, viewed along the cutting plane shown in FIG. 55.

FIG. 58 is an expanded side sectional view of the arc-and-loop suture grasper of FIG. 57.

FIG. 59 is an expanded perspective view of the arc-and-loop suture grasper of FIG. 51, in which the suture retrieval needle is partially transparent as shown.

FIG. 60 is a perspective view of the arc-and-loop suture grasper of FIG. 51, in which the arced grasper wire is in the second position, and the arc-and-loop suture grasper is exposing the grasper wire loop from the needle lumen.

FIG. 61 is a side view of the arc-and-loop suture grasper of FIG. 60.

FIG. 62 is a top view of the arc-and-loop suture grasper of FIG. 60.

FIG. 63 is a front view of the arc-and-loop suture grasper of FIG. 60.

FIG. 64 is a side sectional view of the arc-and-loop suture grasper of FIG. 60, viewed along the cutting plane shown in FIG. 63.

FIG. 65 is an expanded side sectional view of the arc-and-loop suture grasper of FIG. 64.

FIG. 66 is an expanded perspective view of the arc-and-loop suture grasper of FIG. 60.

FIG. 67 is a perspective view of the suture retrieval needle of the arc-and-loop suture grasper of FIG. 51.

FIG. 68 is a perspective view of the retriever body of the arc-and-loop suture grasper of FIG. 51.

FIG. 69 is a side view of the arced grasper wire of the arc-and-loop suture grasper of FIG. 51.

FIG. 70 is a top view of the looped grasper wire of the arc-and-loop suture grasper of FIG. 51.

FIG. 71 is a side view of a grasper ferrule of the arc-and-loop suture grasper of FIG. 51.

FIG. 72 is a perspective view of an arced grasper wire, a looped grasper wire, and a grasper ferrule of the arc-and-loop suture grasper of FIG. 51.

FIG. 73 is a perspective view of a proximal hub of the arc-and-loop suture grasper of FIG. 51.

FIG. 74 is a front view of the proximal hub of FIG. 73.

FIG. 75 is a back view of the proximal hub of FIG. 73.

FIG. 76 is a side view of a spring of the arc-and-loop suture grasper of FIG. 51.

FIG. 77 is a perspective view of an end cap of the arc-and-loop suture grasper of FIG. 51.

FIG. 78 is a front view of the end cap of FIG. 77.

FIG. 79 is a back view of the end cap of FIG. 77.

FIG. 80 is a perspective view of a spring hat of the arc-and-loop suture grasper of FIG. 51.

FIG. 81 is a front view of the spring hat of FIG. 80.

FIG. 82 is a back view of the spring hat of FIG. 80.

FIG. 83 is a side view of a stay collar of the arc-and-loop suture grasper of FIG. 51.

FIG. 84 is a front view of the stay collar of FIG. 83.

FIG. 85 is a back view of the stay collar of FIG. 83.

FIG. 86 is a side view of a retriever body, a proximal hub, an end cap, a spring hat, and a stay collar of the arc-and-loop suture grasper of FIG. 51.

FIG. 87 is a side view of a retriever body, a proximal hub, an end cap, a spring hat, a stay collar, a spring, a suture retrieval needle, an arced grasper wire, and a looped grasper wire of the arc-and-loop suture grasper of FIG. 51, in which the arced grasper wire is in the first position and thus the looped grasper wire is sequestered within the needle lumen.

FIG. 88 is a side view of a retriever body, a proximal hub, an end cap, a spring hat, a stay collar, a spring, a suture retrieval needle, an arced grasper wire, and a looped grasper wire of the arc-and-loop suture grasper of FIG. 51, in which the arced grasper wire is in the second position and thus the looped grasper wire is exposed from the needle lumen.

FIG. 89 is a perspective view of another embodiment of an arc-and-loop suture grasper for grasping a suture comprising a suture retrieval needle, a retriever body, an arced grasper wire, and a looped grasper wire as disclosed herein, in which the arced grasper wire is in the first position, and the arc-and-loop suture grasper is sequestering the grasper wire loop within the needle lumen.

FIG. 90 is a photo of a prototype of an arc-and-loop suture grasper as disclosed herein in which the arced grasper wire is in the second position. A suture is also shown.

FIG. 91 is a photo of the prototype of the arc-and-loop suture grasper of FIG. 90, in which the suture fits between the arced grasper wire and the grasper wire loop beyond the needle lumen.

FIG. 92 is a photo of the prototype of the arc-and-loop suture grasper of FIG. 90, in which the arced grasper wire is in a position intermediate between the second position and the first position, and a suture is disposed between the arced grasper wire and the grasper wire loop.

FIG. 93 is a photo of the prototype of the arc-and-loop suture grasper of FIG. 90, in which the arced grasper wire is in the first position, the arced grasper wire has arced toward the grasper wire loop, the grasper wire loop is sequestered, and the suture is being grasped between the arced grasper wire and the grasper wire loop within the needle lumen.

### DETAILED DESCRIPTION

Our arc-and-loop suture grasper comprising a suture retrieval needle, a retriever body, an arced grasper wire, and a looped grasper wire as disclosed herein provides many of the advantages of previous multi-arm suture passers and magnet-assisted suture passers, as well as new advantages, while avoiding many of the disadvantages of previous multi-arm suture passers and magnet-assisted suture passers.

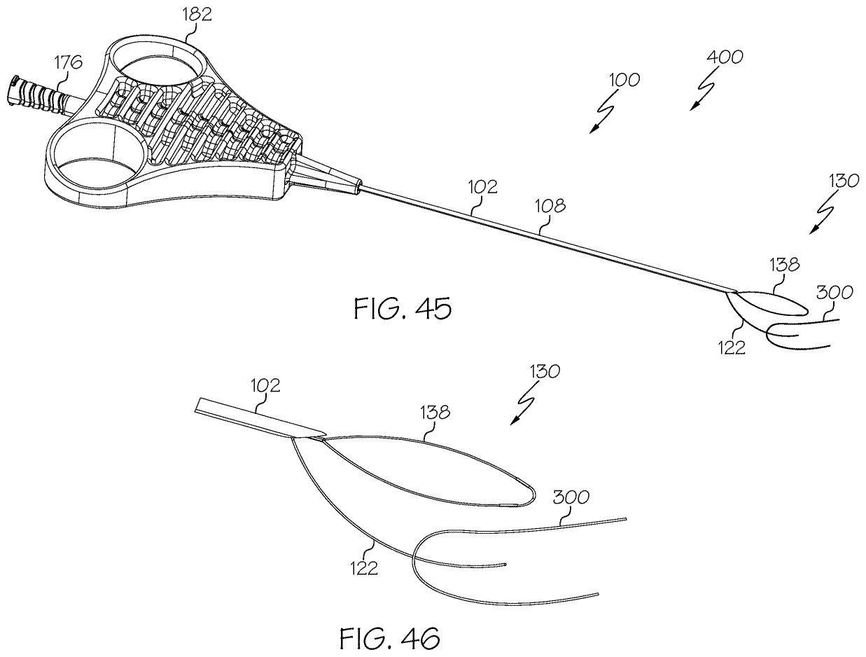

The advantages of our arc-and-loop suture grasper can be appreciated by considering an example embodiment including a grasper wire loop of a looped grasper wire, such as a closed Nitinol wire loop, and an arced grasper wire, such as a Nitinol wire arc, that extend from and retract into a suture retrieval needle, such as a hypodermic needle, as shown in FIG. 45 and FIG. 46. The Nitinol wire loop extends straight out from the hypodermic needle, while the Nitinol wire arc bends away from Nitinol wire loop.

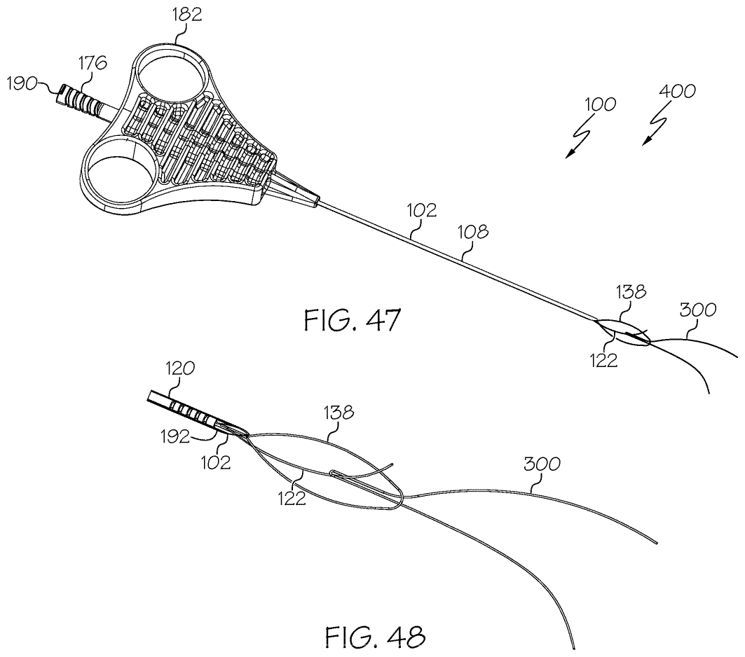

This configuration allows for capturing a suture between the Nitinol wire arc and the Nitinol wire loop. This is because as the Nitinol wire arc and the Nitinol wire loop begin to be retracted into the hypodermic needle, the Nitinol wire arc automatically bends and feeds through the eye of the closed Nitinol wire loop. This is shown in FIG. 47 and FIG. 48. This mechanism lightly grips the suture as the suture and Nitinol wires are further retracted into the hypodermic needle.

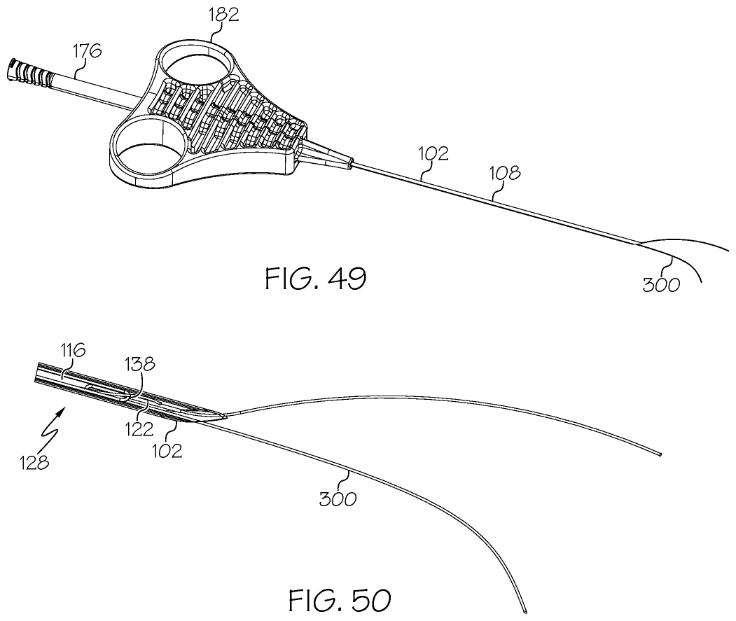

When the Nitinol wires are fully retracted into the hypodermic needle, the Nitinol wire arc weaves under the Nitinol wire loop, essentially creating a secondary closed loop between the Nitinol wire arc and Nitinol wire loop. This is shown in FIG. 49 and FIG. 50.

The key for this mechanism to work is for the bent Nitinol wire arc to be short enough to enter the Nitinol wire loop during the first stage of the retraction. However, when the Nitinol wires are fully retracted into the hypodermic needle, the relatively “straightened” Nitinol arc wire is actually longer than the collapsed nitinol wire loop. The weaving of the Nitinol wire arc through, under, and past the Nitinol wire loop effectively locks the suture in place with very thin, flexible wires while the Nitinol wires remain fully retracted.

The suture can be released from the arc-and-loop suture grasper by extending the Nitinol wire loop and a Nitinol wire arc again.

Thus, our arc-and-loop suture grasper can be used to capture and release sutures multiple times.

This mechanism of suture grasping/retention provides many advantages compared to previous suture passers.

Unlike the pincer-type suture passers discussed above, which rely on force generated by the end effector to grasp/pinch the suture, our arc-and-loop suture grasper essentially relies on a “woven loop” and the ultimate tensile strength of the Nitinol wires. The Nitinol wires, by design, can be very thin. For example, we used 0.008 inch (0.20 mm) diameter Nitinol wire in our prototypes. This allows making suture passers with very small hypodermic needles. For example, we used 18 gauge (1.2 mm) hypodermic needles on our prototypes.

Since the Nitinol wires are thin and flexible, the hypodermic needle of our suture passer can be bent at different angles and the Nitinol wires will still be able to move through the inner lumen of the hypodermic needle. Using a suture passer having a hypodermic needle with a curved tip will help surgeons navigate difficult features within the surgical field.

Our arc-and-loop suture grasper can be provided with a patent lumen extending from its proximal end to its distal end, through the retriever body and the suture retrieval needle.

This can be accomplished, for example, by using the Nitinol crimping method of assembly disclosed in Applied Medical Technology, Inc.'s international application PCT/US2022/050444, filed Nov. 18, 2022. The patent lumen can allow the administration of fluids, such as contrast for fluoroscopy/X-rays, local administration of medication, application of liquid bio-adhesives, and/or water/saline for hydro-dissection techniques. The patent lumen also can allow for the withdrawal of fluids or air. A Luer-tip syringe filled with water/saline can be connected to the arc-and-loop suture grasper at the proximal end of the lumen. When so connected, bubbles appearing in the water/saline in the syringe will indicate that the distal end of the arc-and-loop suture passer has entered an insufflated surgical site.

Our arc-and-loop suture grasper has a much larger grasping target area in comparison to pincer-type and snare-type suture passers. This will make it easier for surgeons to manipulate and grasp a suture within the surgical field, which is typically in two-dimensional vision in laparoscopic surgeries.

Our arc-and-loop suture grasper can grasp sutures anywhere along the length of the suture.

The closed Nitinol wire loop of our arc-and-loop suture grasper can be used as a standard snare. This allows the device to manipulate the two ends of the suture, much like snare-type suture passers having a loop, when required during a procedure.

The thin Nitinol wires are very flexible, and easily spring back to their pre-deformed shapes. This will allow our arc-and-loop suture grasper to be used in surgical sites with limited space. Moreover, in contrast to pincer-type suture passers, the Nitinol wire loop and Nitinol wire arc of our arc-and-loop suture grasper will not damage delicate surrounding tissues.

Our arc-and-loop suture grasper can utilize a wide variety of suture types, styles, and sizes. In contrast to magnet-assisted suture passers, our arc-and-loop suture grasper does not require specialized sutures that include a suture magnet.

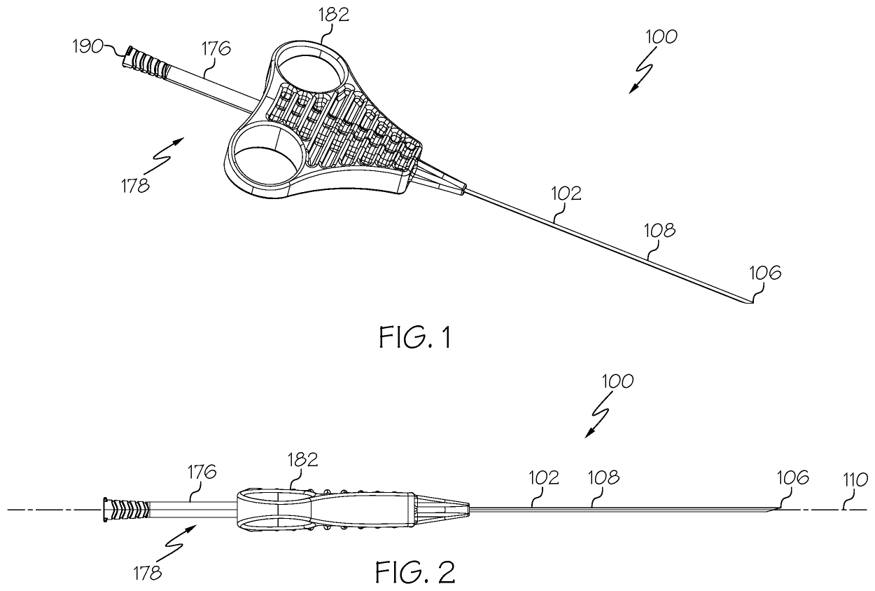

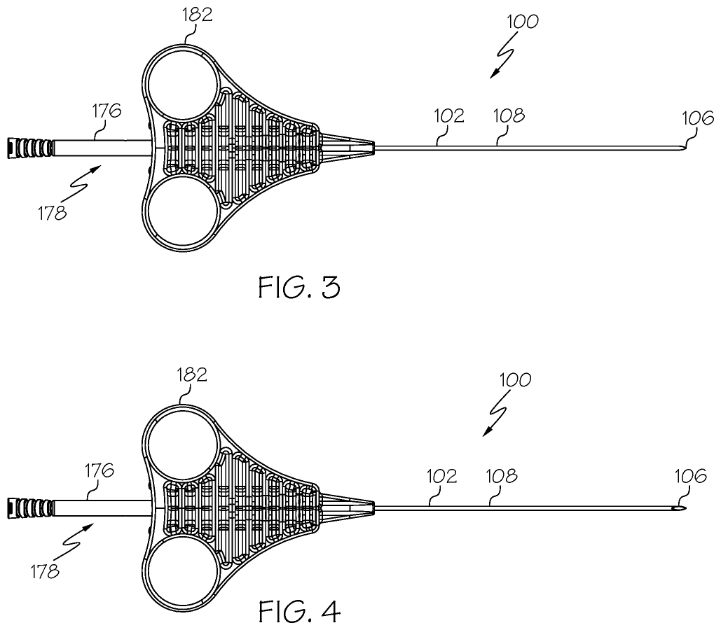

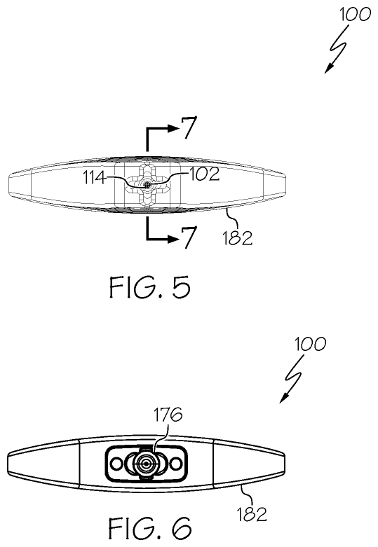

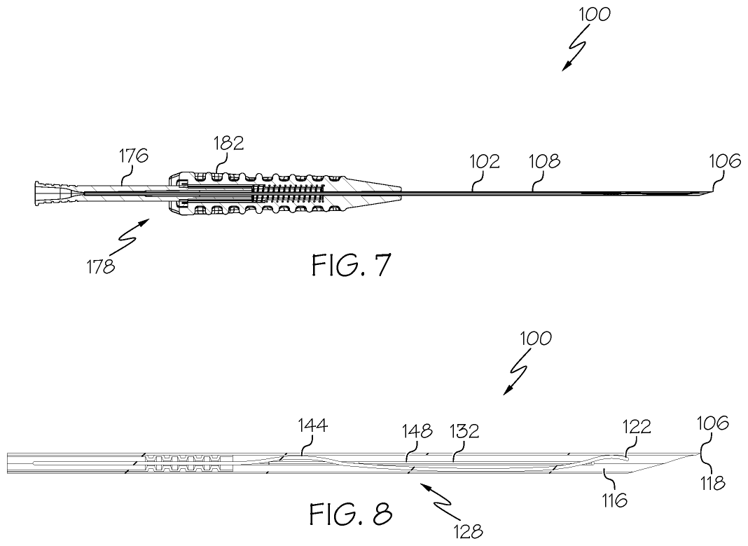

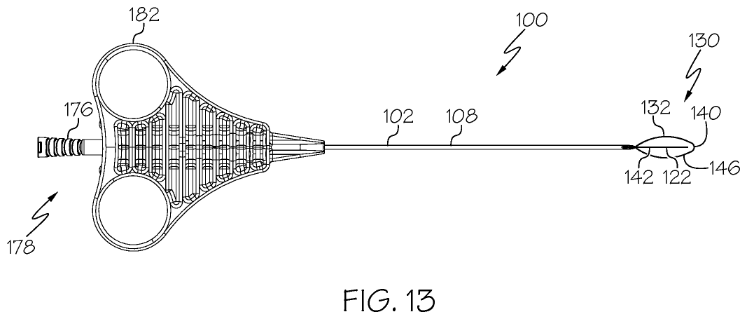

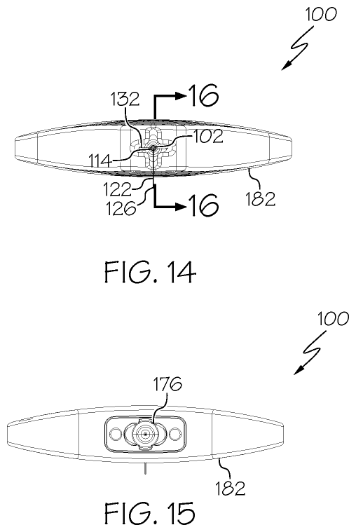

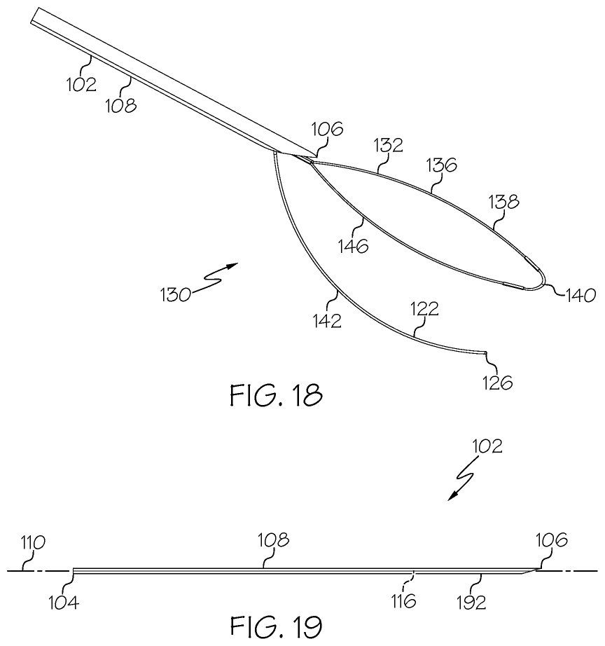

An example embodiment of an arc-and-loop suture grasper 100 for grasping a suture 300 is shown in FIGS. 1-18, in which FIGS. 1-9 show the arc-and-loop suture grasper 100 with an arced grasper wire 122 in a first position 128, and the arc-and-loop suture grasper 100 sequestering a grasper wire loop 138 within a needle lumen 116, and FIGS. 10-18 show the arc-and-loop suture grasper 100 with the arced grasper wire 122 in the second position 130, and the arc-and-loop suture grasper 100 exposing the grasper wire loop 138 from the needle lumen 116.

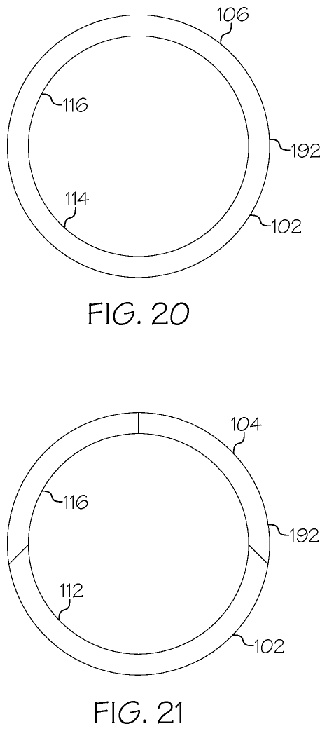

As shown in FIGS. 19-21, with reference to FIGS. 1-18, the arc-and-loop suture grasper 100 comprises a suture retrieval needle 102. The suture retrieval needle 102 comprises a proximal end 104, a distal end 106, and a needle body 108 extending therebetween. The needle body 108 defines a needle body axis 110 between the proximal end 104 and the distal end 106 of the suture retrieval needle 102. The needle body 108 has a proximal hole 112, a distal hole 114, and a needle lumen 116 extending therebetween along the needle body axis 110.

The suture retrieval needle 102 can be, for example, a hypodermic needle. For example, the suture retrieval needle 102 can be an introducer needle designed for introducing guide wires into a vessel, applied here as the suture retrieval needle 102.

The suture retrieval needle 102 should have a size selected for suitability for the patient and the procedure and taking into account sizes of the sutures, arced grasper wire 122, and looped grasper wire 132 to be used. The suture retrieval needle 102 can be, for example, a 24-gauge (0.51 mm) needle, a 21-gauge (0.80 mm) needle, an 18-gauge (1.2 mm) needle, a 17-gauge (1.4 mm) needle, a 16-gauge (1.6 mm) needle, or a 14-gauge (2.0 mm) needle.

As shown in FIGS. 1-18, in some embodiments, the suture retrieval needle 102 is straight. In accordance with these embodiments, the needle body axis 110 thereby is straight. Alternatively, in some embodiments the suture retrieval needle 102 is curved. In accordance with these embodiments, the needle body axis 110 thereby is curved. In some embodiments, one or more portions of the suture retrieval needle 102 can be straight, and one or more portions can be curved. For example, in some embodiments, the suture retrieval needle 102 includes a curve at or near its distal end 106 but otherwise is straight. In accordance with these embodiments, the needle body axis 110 also includes a curve at or near the distal end 106 of the suture retrieval needle 102, but otherwise is straight.

As shown in FIG. 8, in some embodiments, the suture retrieval needle 102 has a sharp tip 118. This can be advantageous for piercing tissue during insertion of the suture retrieval needle 102 into a patient.

As shown in FIGS. 22-24, with reference to FIGS. 1-18, the arc-and-loop suture grasper 100 also comprises a retriever body 120. The retriever body 120 is disposed within the needle lumen 116 and translatable therein along the needle body axis 110. The retriever body 120 can be made from a polymer, such as nylon, polyether ether ketone, polycarbonate, acrylonitrile butadiene styrene, co-polyester, polypropylene, acetal (also termed polyoxymethylene or POM), or polyurethane, or a metal, such as stainless steel or Nitinol.

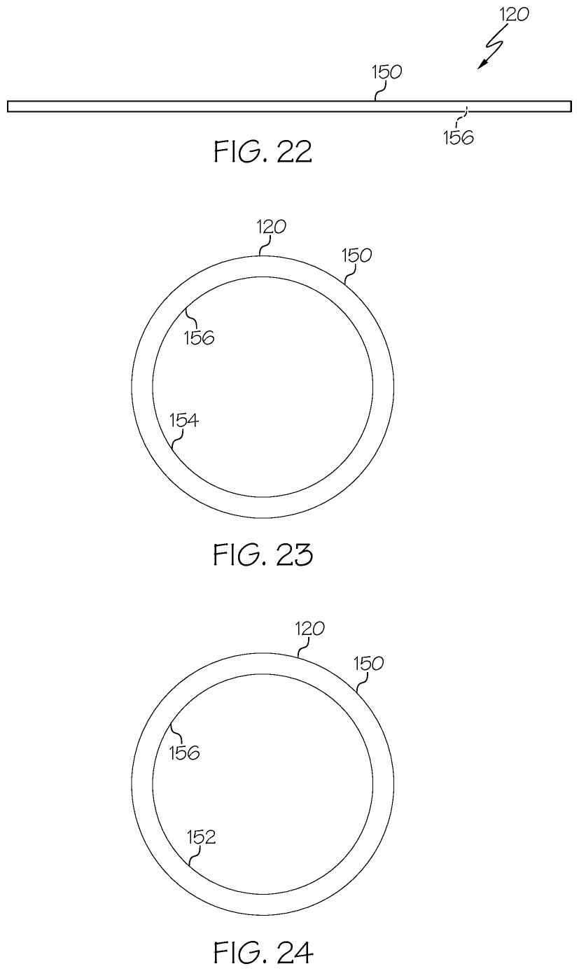

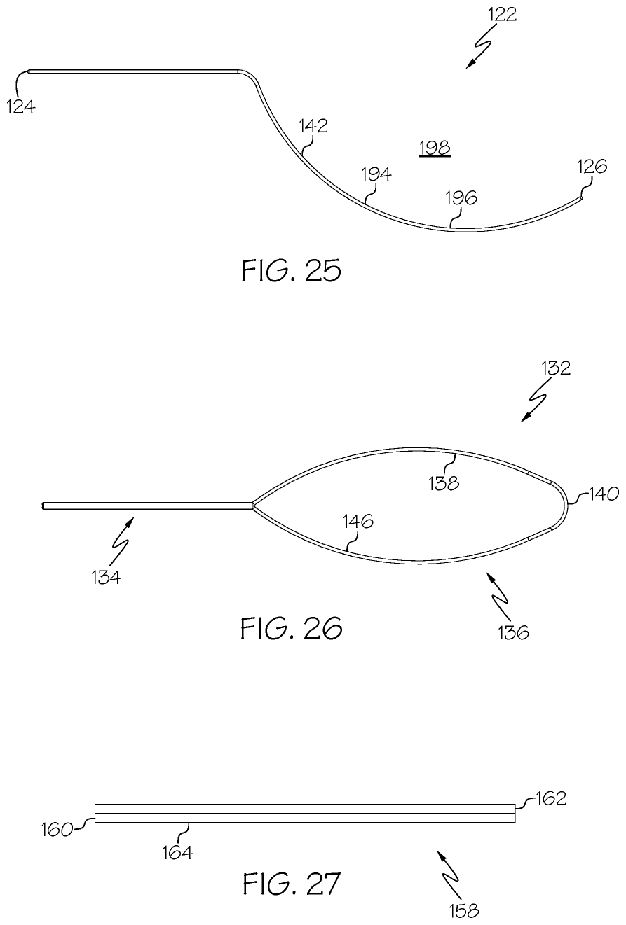

As shown in FIG. 25, with reference to FIGS. 1-18, the arc-and-loop suture grasper 100 also comprises an arced grasper wire 122. The arced grasper wire 122 has a proximal end 124 and a distal end 126. The proximal end 124 of the arced grasper wire 122 is fixedly disposed within the retriever body 120.

The arced grasper wire 122 extends distally from the retriever body 120 and is reversibly moveable between a first position 128 and a second position 130, as discussed below.

In some embodiments, the arced grasper wire 122 is formed from one or more of a shape memory alloy, titanium, stainless steel, or a monofilament polymer. The shape memory alloy can be, for example, Nitinol.

The arced grasper wire 122 should have a size selected for suitability for the patient and the procedure and taking into account sizes of the sutures, suture retrieval needle 102, and looped grasper wire 132 to be used. The arced grasper wire 122 can have a diameter of, for example, 0.001 inches (0.025 mm) to 0.016 inches (0.40 mm), 0.002 inches (0.051 mm) to 0.014 inches (0.36 mm), 0.003 inches (0.076 mm) to 0.012 inches (0.30 mm), 0.004 (0.10 mm) to 0.010 inches (0.25 mm), or 0.005 (0.13 mm) to 0.008 inches (0.20 mm), among other diameters. Thus, for example, the arced grasper wire 122 can have a diameter of 0.001 inches (0.025 mm), 0.002 inches (0.051 mm), 0.003 inches (0.076 mm), 0.004 inches (0.10 mm), 0.005 inches (0.13 mm), 0.006 inches (0.15 mm), 0.007 inches (0.18 mm), 0.008 inches (0.20 mm), 0.009 inches (0.23 mm), 0.010 inches (0.25 mm), 0.011 inches (0.28 mm), 0.012 inches (0.30 mm), 0.013 inches (0.33 mm), 0.014 inches (0.36 mm), 0.015 inches (0.38 mm), or 0.016 inches (0.40 mm), among other diameters. Diameters less than 0.001 inches (0.025 mm) or greater than 0.016 inches (0.40 mm) may be suitable too.

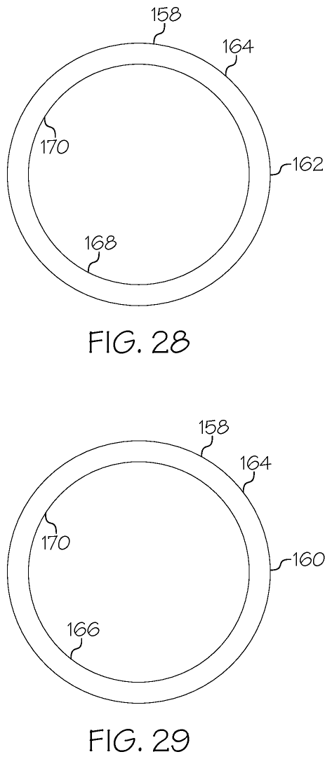

As shown in FIG. 26, with reference to FIGS. 1-18, the arc-and-loop suture grasper 100 also comprises a looped grasper wire 132. The looped grasper wire 132 comprises a proximal portion 134 and a distal portion 136. The proximal portion 134 of the looped grasper wire 132 is fixedly disposed within the retriever body 120. The distal portion 136 of the looped grasper wire 132 forms a grasper wire loop 138. The grasper wire loop 138 has a distal-most loop point 140. The looped grasper wire 132 extends distally from the retriever body 120. The arc-and-loop suture grasper 100 sequesters the grasper wire loop 138 within the needle lumen 116 when the arced grasper wire 122 is in the first position 128 and exposes the grasper wire loop 138 from the needle lumen 116 when the arced grasper wire 122 is in the second position 130, as discussed below.

In some embodiments, the looped grasper wire 132 is formed from one or more of a shape memory alloy, titanium, stainless steel, or a monofilament polymer. The shape memory alloy can be, for example, Nitinol.

The looped grasper wire 132 should have a size selected for suitability for the patient and the procedure and taking into account sizes of the sutures, suture retrieval needle 102, and arced grasper wire 122 to be used. The looped grasper wire 132 can have a diameter of, for example, 0.001 inches (0.025 mm) to 0.016 inches (0.40 mm), 0.002 inches (0.051 mm) to 0.014 inches (0.36 mm), 0.003 inches (0.076 mm) to 0.012 inches (0.30 mm), 0.004 (0.10 mm) to 0.010 inches (0.25 mm), or 0.005 (0.13 mm) to 0.008 inches (0.20 mm), among other diameters. Thus, for example, the looped grasper wire 132 can have a diameter of 0.001 inches (0.025 mm), 0.002 inches (0.051 mm), 0.003 inches (0.076 mm), 0.004 inches (0.10 mm), 0.005 inches (0.13 mm), 0.006 inches (0.15 mm), 0.007 inches (0.18 mm), 0.008 inches (0.20 mm), 0.009 inches (0.23 mm), 0.010 inches (0.25 mm), 0.011 inches (0.28 mm), 0.012 inches (0.30 mm), 0.013 inches (0.33 mm), 0.014 inches (0.36 mm), 0.015 inches (0.38 mm), or 0.016 inches (0.40 mm), among other diameters. Diameters less than 0.001 inches (0.025 mm) or greater than 0.016 inches (0.40 mm) may be suitable too.

The arced grasper wire 122 and the looped grasper wire 132 are more flexible than the needle body 108. This allows the arced grasper wire 122 and the looped grasper wire 132 to deform when retracted into the needle lumen 116 of the suture retrieval needle 102. For suture retrieval needles 102 in which one or more portions of the suture retrieval needle 102 are curved, e.g., a suture retrieval needle 102 that includes a curve at or near its distal end 106 but otherwise is straight, this also allows the arced grasper wire 122 and the looped grasper wire 132 to elastically deform around the curve of the suture retrieval needle 102.

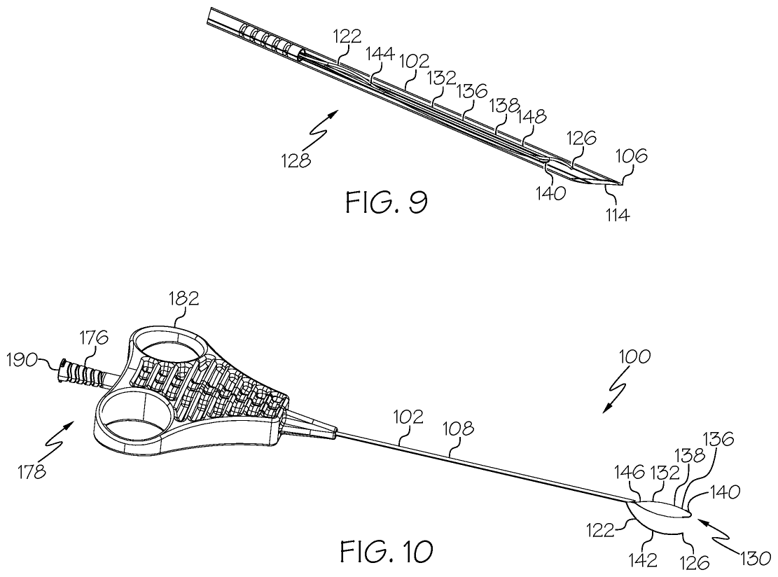

As shown in FIG. 9, when the arced grasper wire 122 is in the first position 128 the arced grasper wire 122 extends through the grasper wire loop 138 and the distal end 126 of the arced grasper wire 122 extends further distally than the distal-most loop point 140 of the grasper wire loop 138.

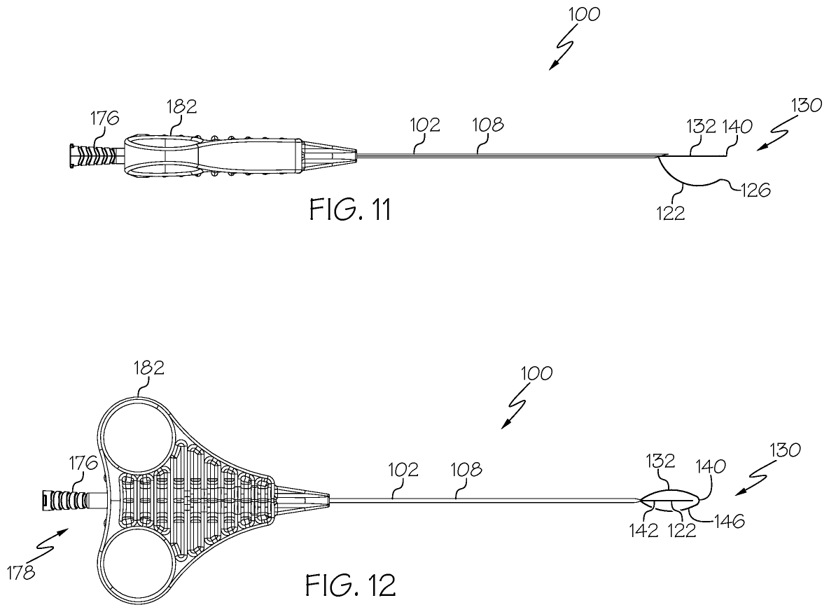

As shown in FIGS. 11-13, FIG. 16, and FIG. 17, when the arced grasper wire 122 is in the second position 130 the arced grasper wire 122 arcs away from the grasper wire loop 138 and the distal-most loop point 140 of the grasper wire loop 138 extends further distally than the distal end 126 of the arced grasper wire 122.

The arced grasper wire 122 is sufficiently short to exit the grasper wire loop 138 as the arced grasper wire 122 is moved from the first position 128 to the second position 130 and the looped grasper wire 132 translates in unison with the arced grasper wire 122, and to enter the grasper wire loop 138 as the arced grasper wire 122 is moved from the second position 130 to the first position 128 and the looped grasper wire 132 translates in unison with the arced grasper wire 122.

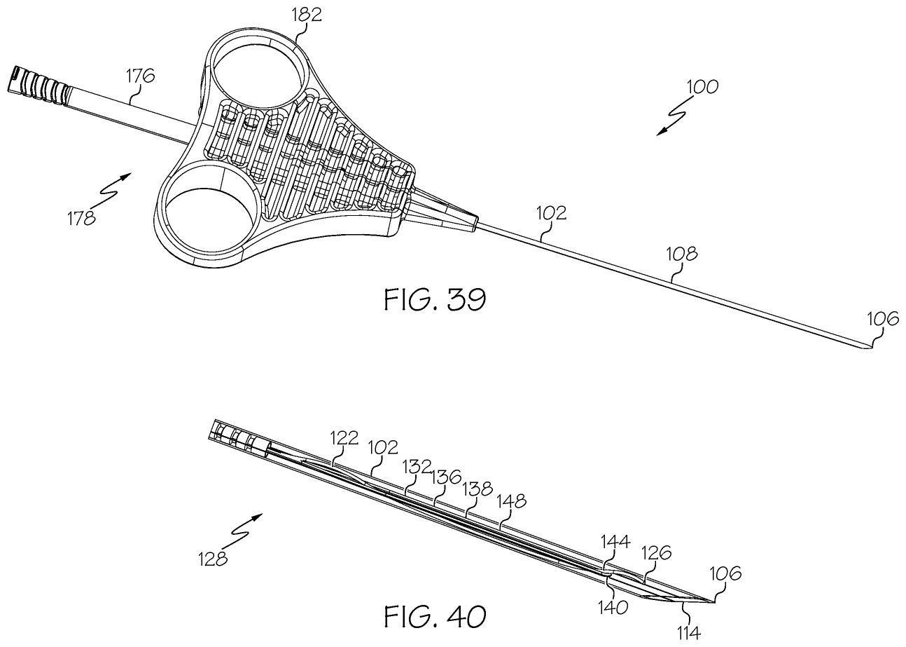

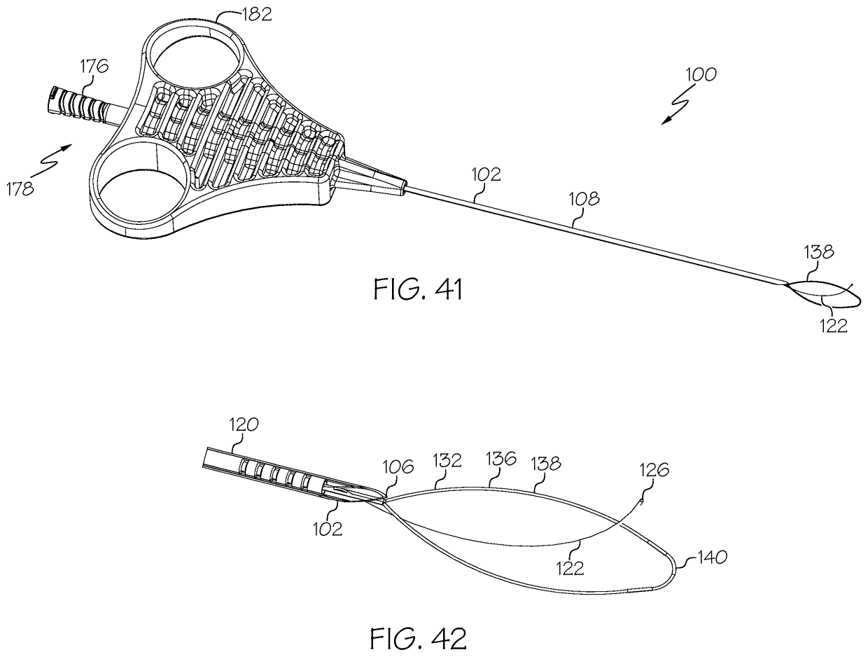

With reference to FIGS. 39-44, translation of the retriever body 120 within the needle lumen 116 in a first direction along the needle body axis 110 causes the arced grasper wire 122 to move from the first position 128 to the second position 130, thereby allowing the arced grasper wire 122 to exit the grasper wire loop 138 and arc away from the grasper wire loop 138, exposing the grasper wire loop 138, and allowing a suture 300 to fit between the arced grasper wire 122 and the grasper wire loop 138 beyond the needle lumen 116. The translation of the retriever body 120 results in translation of the arced grasper wire 122 and the looped grasper wire 132, including the grasper wire loop 138, in unison. As noted above, the arced grasper wire 122 is sufficiently short to exit the grasper wire loop 138 as the arced grasper wire 122 is moved from the first position 128 to the second position 130 and the looped grasper wire 132 translates in unison with the arced grasper wire 122.

The translation of the retriever body 120 in the first direction can be translation of the retriever body 120 within the needle lumen 116 in a direction from the proximal end 104 of the suture retrieval needle 102 toward the distal end 106 of the suture retrieval needle 102.

When the arced grasper wire 122 is in the first position 128 the arced grasper wire 122 can extend through the grasper wire loop 138 and the distal end 126 of the arced grasper wire 122 can extend further distally than the distal-most loop point 140 of the grasper wire loop 138 based on the arced grasper wire 122 having been deformed from a relaxed non-deformed shape 142 to a constrained deformed shape 144 due to being constrained within the needle lumen 116. Such translation of the retriever body 120 within the needle lumen 116 in the first direction can allow a substantial portion of the arced grasper wire 122 to emerge distally from the needle lumen 116, thus allowing the arced grasper wire 122 to arc away from the grasper wire loop 138 by transitioning from its constrained deformed shape 144 to its relaxed non-deformed shape 142.

Also when the arced grasper wire 122 is in the first position 128 the grasper wire loop 138 can be disposed entirely inside the needle lumen 116, and thus sequestered within the needle lumen 116, based on the looped grasper wire 132 having been deformed from a relaxed non-deformed shape 146 to a constrained deformed shape 148 also due to being constrained within the needle lumen 116. Such translation of the retriever body 120 within the needle lumen 116 in the first direction can move the grasper wire loop 138 from inside the needle lumen 116 to outside the needle lumen 116, thus exposing the grasper wire loop 138 and allowing the looped grasper wire 132 to transition from its constrained deformed shape 148 to its relaxed non-deformed shape 146.

Once the arced grasper wire 122 has arced away from the grasper wire loop 138, and the grasper wire loop 138 has been exposed, a suture 300 can fit between the arced grasper wire 122 and the grasper wire loop 138 beyond the needle lumen 116, for example based on positioning the arced grasper wire 122 and the grasper wire loop 138 to encompass the suture 300.

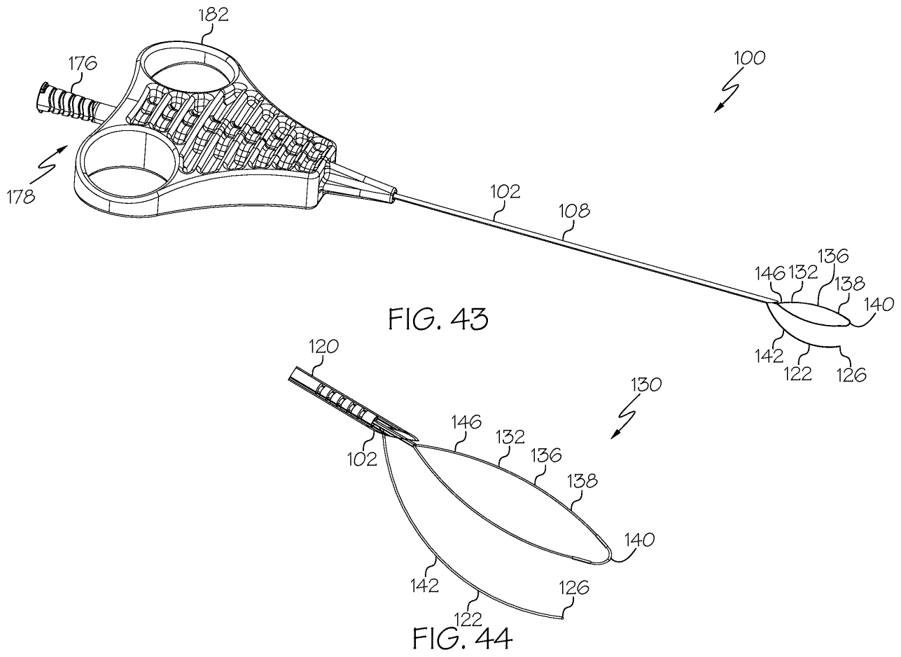

With reference to FIGS. 43-50, translation of the retriever body 120 within the needle lumen 116 in a second direction opposite the first direction along the needle body axis 110 causes the arced grasper wire 122 to move from the second position 130 to the first position 128, thereby causing the arced grasper wire 122 to arc toward the grasper wire loop 138 and enter the grasper wire loop 138, sequestering the grasper wire loop 138, and grasping the suture 300 between the arced grasper wire 122 and the grasper wire loop 138 within the needle lumen 116. As noted above, the translation of the retriever body 120 results in translation of the arced grasper wire 122 and the looped grasper wire 132, including the grasper wire loop 138, in unison. Also as noted, the arced grasper wire 122 is sufficiently short to enter the grasper wire loop 138 as the arced grasper wire 122 is moved from the second position 130 to the first position 128 and the looped grasper wire 132 translates in unison with the arced grasper wire 122.

The translation of the retriever body 120 in the second direction can be translation of the retriever body 120 within the needle lumen 116 in a direction from the distal end 106 of suture retrieval needle 102 toward the proximal end 104 of the suture retrieval needle 102.

Such translation of the retriever body 120 within the needle lumen 116 in the second direction can return a substantial portion of the arced grasper wire 122 proximally into the needle lumen 116, causing the arced grasper wire 122 to collapse and return to its constrained deformed shape 144 as the arced grasper wire 122 becomes constrained again within the needle lumen 116.

Such translation also can move the grasper wire loop 138 from outside the needle lumen 116 to inside the needle lumen 116, also causing the looped grasper wire 132 to collapse and return to its constrained deformed shape 148 as the looped grasper wire 132 becomes constrained again within the needle lumen 116, thus sequestering the grasper wire loop 138 again.

By positioning the arced grasper wire 122 and the looped grasper wire 132 to encompass a suture 300 when the arced grasper wire 122 is in the second position 130, and then translating the retriever body 120 within the needle lumen 116 in a second direction opposite the first direction along the needle body axis 110, the arced grasper wire 122 can capture the suture 300 between the arced grasper wire 122 and the looped grasper wire 132 as the arced grasper wire 122 begins to transition from its relaxed non-deformed shape 142 to its constrained deformed shape 144 and returns to extending through the grasper wire loop 138.

As the arced grasper wire 122 translates through positions intermediate between the second position 130 and the first position 128, the arced grasper wire 122 can lightly grip the suture 300 and pull the suture 300 into the needle lumen 116.

Upon returning to the first position 128, the arced grasper wire 122 again extends through the grasper wire loop 138 and the distal end 126 of the arced grasper wire 122 again extends further distally than the distal-most loop point 140 of the grasper wire loop 138, as the arced grasper wire 122 and the looped grasper wire 132 have returned to their respective constrained deformed shapes 144, 148.

As described above, this effectively locks the suture 300 in place between the arced grasper wire 122 and the looped grasper wire 132. This is because having the arced grasper wire 122 extend through the grasper wire loop 138 and having the distal end 126 of the arced grasper wire 122 extend further distally than the distal-most loop point 140 of the grasper wire loop 138 results in weaving of the arced grasper wire 122 through, under, and past the grasper wire loop 138 of the looped grasper wire 132, essentially creating a secondary closed loop between the arced grasper wire 122 and the looped grasper wire 132. This effectively locks the suture 300 in place even when using an arced grasper wire 122 and a looped grasper wire 132 made of very thin, flexible wires, for example from a shape memory alloy, such as Nitinol, or titanium, stainless steel, or a monofilament polymer.

The suture 300 can be released from the arc-and-loop suture grasper 100 by translation of the retriever body 120 within the needle lumen 116 in the first direction along the needle body axis 110 again, causing the arced grasper wire 122 to move from the first position 128 to the second position 130, thereby allowing the arced grasper wire 122 to arc away from the grasper wire loop 138, and exposing the grasper wire loop 138 again.

Thus, our arc-and-loop suture grasper 100 can be used to capture and release sutures 300 multiple times.

This provides many advantages, as discussed above.

Considering the arc-and-loop suture grasper 100 further, as shown in FIGS. 22-24, with reference to FIGS. 1-18, in some embodiments the retriever body 120 comprises a retriever tube 150 having a proximal hole 152, a distal hole 154, and a retriever tube lumen 156 extending therebetween. In these embodiments, the retriever tube 150 is partially disposed within the needle lumen 116 and translatable therein along the needle body axis 110. Also in these embodiments, the proximal hole 152 of the retriever tube 150 is in fluid communication with the distal hole 114 of the needle body 108 through the retriever tube lumen 156 and the needle lumen 116.

This can be advantageous by allowing recovery of liquids and/or gasses from a surgical site of a patient and/or delivery of contrast agents to the surgical site through the retriever tube lumen 156. Providing the ability to exchange fluids through the retriever tube lumen 156, and thus through the retriever body 120 and the arc-and-loop suture grasper 100 broadens the applicability of the arc-and-loop suture grasper 100 for use with interventional techniques that employ fluid exchange to confirm the intracorporeal position of the distal end 106 of the suture retrieval needle 102 of the arc-and-loop suture grasper 100. For example, a need exists within the field of interventional radiology for the ability to confirm the location of cannulas inside the gastric lumens of patients during gastropexy. With the arc-and-loop suture grasper 100 comprising the retriever body 120 with the retriever tube lumen 156, following introduction of the suture retrieval needle 102 into a patient, aspiration of a small amount of stomach juice or air can be used to confirm the intraluminal position of the distal end 106 of the suture retrieval needle 102. Alternatively, a small amount of liquid radiographic contrast agent can be injected through the retriever tube lumen 156 of the retriever body 120 into the gastric lumen of the patient, allowing the intraluminal position to be confirmed through radiographic imaging.

In some of the embodiments in which the retriever body 120 comprises a retriever tube 150 having a proximal hole 152, a distal hole 154, and a retriever tube lumen 156 extending therebetween, the retriever tube 150 comprises stainless steel. Also in some of these embodiments, the retriever tube lumen 156 is the only lumen of the retriever tube 150. The arc-and-loop suture grasper 100 comprising a retriever tube 150 being made from a hard metal such as stainless steel and having only one lumen and provides advantages of greater structural integrity, more consistent operative feel, broader chemical compatibility, and greater ease of manufacture.

In some of the embodiments in which the retriever body 120 comprises a retriever tube 150 having a proximal hole 152, a distal hole 154, and a retriever tube lumen 156 extending therebetween, translation of the retriever tube 150 within the needle lumen 116 is limited to a straight path as the translation of the retriever tube 150 causes the arced grasper wire 122 to move between a first position 128 and a second position 130. Limiting translation of the retriever tube 150 within the needle lumen 116 to a straight path as the translation of the retriever tube 150 causes the arced grasper wire 122 to move between the first position 128 and the second position 130 can be advantageous because it allows use of a retriever tube 150 that is made of a rigid material, such as stainless steel, while also allowing use of a suture retrieval needle 102 that includes a curve at or near its distal end 106 but otherwise is straight. In accordance with these embodiments, the arced grasper wire 122 and the looped grasper wire 132 can be made to sufficient lengths and with sufficient flexibilities to pass through the needle lumen 116 at the curve of the suture retrieval needle 102 without the retriever tube 150 needing to do so.

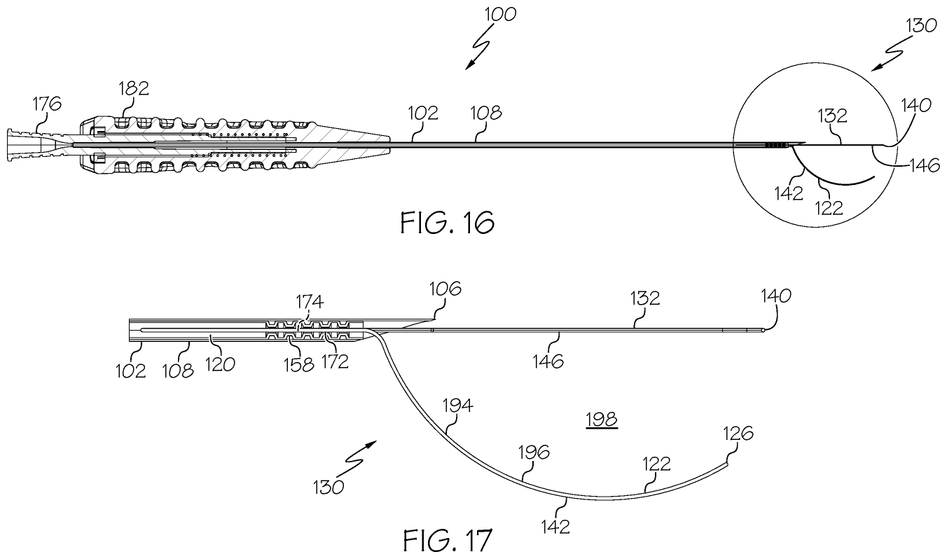

With reference to FIG. 11, FIG. 16, and FIG. 17, in some embodiments the arced grasper wire 122 comprises a distal portion 194 that forms an arcuate curve 196 that defines an arcuate-curve interior 198 between the grasper wire 122 and the grasper wire loop 138 when the arced grasper wire 122 is in the second position 130. The arcuate curve 196 can be, for example, an arc of a portion of a circle or an oval, among other generally round shapes. In accordance with these embodiments, as the arced grasper wire 122 is moved from the first position 128 to the second position 130 and transitions from the constrained deformed shape 146 to the relaxed non-deformed shape 142, the distal portion 194 of the arced grasper wire 122 reversibly arcs to form the arcuate curve 196 that defines the arcuate-curve interior 198 between the grasper wire 122 and the grasper wire loop 138. This can be advantageous for facilitating the arced grasper wire 122 exiting the grasper wire loop 138 as the arced grasper wire 122 is moved from the first position 128 to the second position 130, and entering the grasper wire loop 138 as the arced grasper wire 122 is moved from the second position 130 to the first position 128.

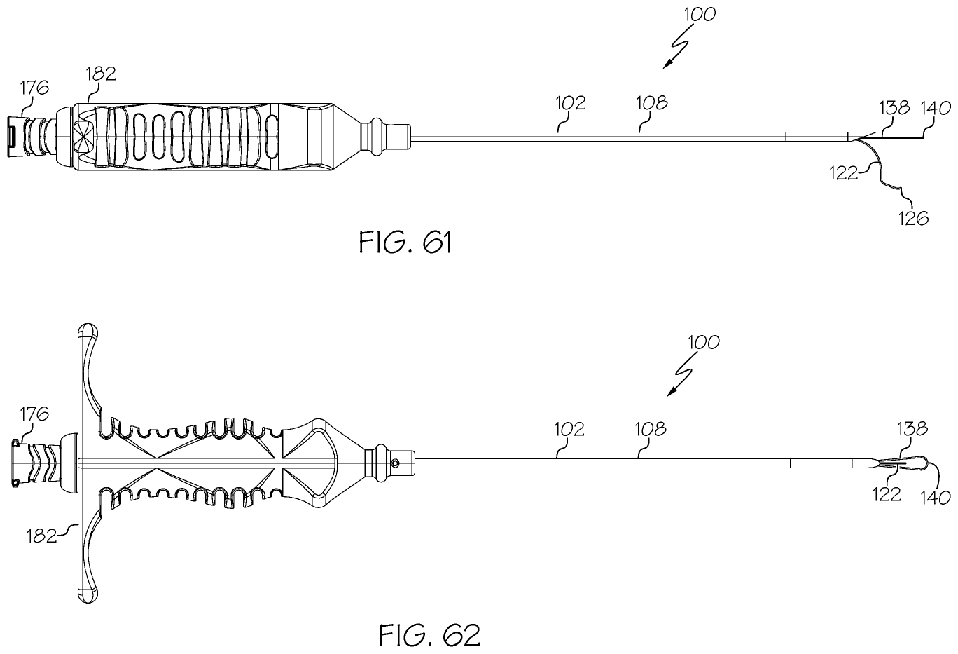

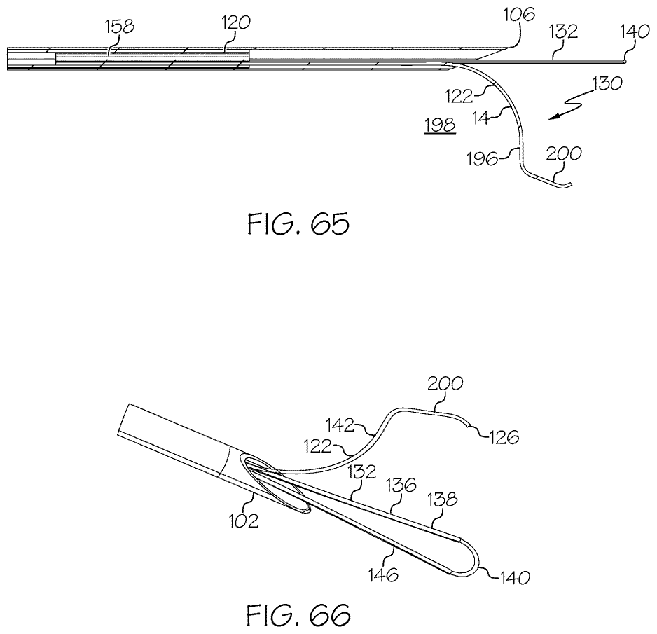

With reference to FIG. 61, FIG. 64, and FIG. 65, in some embodiments the arced grasper wire 122 comprises a distal portion 194 that forms an arcuate curve 196 that defines an arcuate-curve interior 198 not between the grasper wire 122 and the grasper wire loop 138 when the arced grasper wire 122 is in the second position 130. Again, the arcuate curve 196 can be, for example, an arc of a portion of a circle or an oval, among other generally round shapes. In accordance with these embodiments, as the arced grasper wire 122 is moved from the first position 128 to the second position 130 and transitions from the constrained deformed shape 146 to the relaxed non-deformed shape 142, the distal portion 194 of the arced grasper wire 122 reversibly arcs to form the arcuate curve 196 that defines the arcuate-curve interior 198 not between the grasper wire 122 and the grasper wire loop 138. This can be advantageous for providing substantial space between the arced grasper wire 122 and the grasper wire loop 138 for capturing a suture 300.

In some embodiments, the arced grasper wire 122 comprises a distal-most portion 200 that extends transversely from a distal portion 194 of the arced grasper wire 122 when the arced grasper wire 122 is in the second position 130. This can be advantageous for capturing a suture 300 between the arced grasper wire 122 and the looped grasper wire 132 as the arced grasper wire 122 begins to transition from its relaxed non-deformed shape 142 to its constrained deformed shape 146 and returns to extending through the grasper wire loop 138.

With reference to FIGS. 27-31, in some of the embodiments in which the retriever body 120 comprises a retriever tube 150 having a proximal hole 152, a distal hole 154, and a retriever tube lumen 156 extending therebetween, the arc-and-loop suture grasper 100 also comprises a grasper ferrule 158. In these embodiments, the grasper ferrule 158 comprises a proximal end 160, a distal end 162, and a ferrule body 164 extending therebetween. The grasper ferrule 158 can be, for example, a metal crimp ferrule made from stainless steel or Nitinol, among other metals, or a hard plastic ferrule made from polycarbonate or polyether ether ketone, among other hard plastics. The ferrule body 164 has a proximal hole 166, a distal hole 168, and a ferrule lumen 170 extending therebetween. The grasper ferrule 158 is fixedly disposed within the retriever tube 150. Accordingly, the grasper ferrule 158 has an outer diameter that is smaller than an inner diameter of the retriever tube 150. The arced grasper wire 122 and the looped grasper wire 132 extend distally from the grasper ferrule 158.

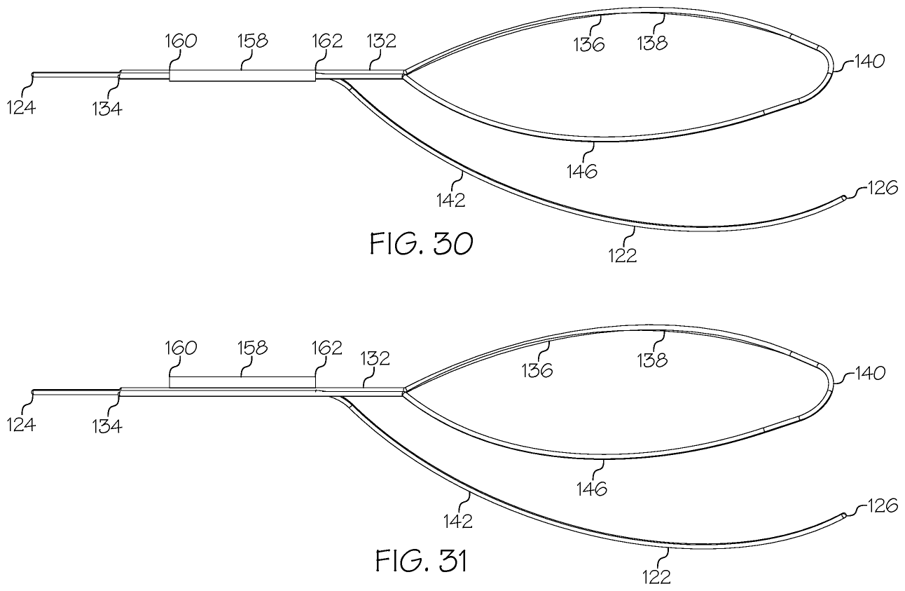

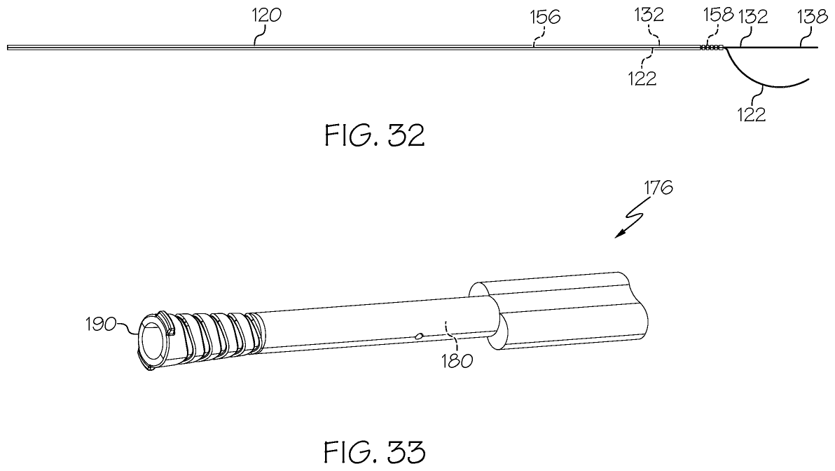

As shown in FIG. 32, with reference to FIG. 30 and FIG. 31, the grasper ferrule 158 can be used to fixedly attach the arced grasper wire 122 and the looped grasper wire 132 to the retriever tube 150 by various approaches.

As shown in FIG. 17, with reference to FIG. 30, in some approaches the grasper ferrule 158 has been crimped onto the arced grasper wire 122 and the looped grasper wire 132 with a crimped portion 172 of the arced grasper wire 122 and a crimped portion 174 of the looped grasper wire 132 being disposed within the ferrule lumen 170 of the grasper ferrule 158, and the retriever tube 150 has been crimped onto the grasper ferrule 158, with the grasper ferrule 158, the crimped portion 172 of the arced grasper wire 122, and the crimped portion 174 of the looped grasper wire 132 being disposed within the retriever tube lumen 156.

With reference to FIG. 31, in other approaches the retriever tube 150 has been crimped onto the grasper ferrule 158, the crimped portion 172 of the arced grasper wire 122, and the crimped portion 174 of the looped grasper wire 132, with the grasper ferrule 158, the crimped portion 172 of the arced grasper wire 122, and the crimped portion 174 of the looped grasper wire 132 being disposed within the retriever tube lumen 156, and with the crimped portion 172 of the arced grasper wire 122 and the crimped portion 174 of the looped grasper wire 132 being disposed adjacent to the grasper ferrule 158 and not within the ferrule lumen 170 of the grasper ferrule 158.

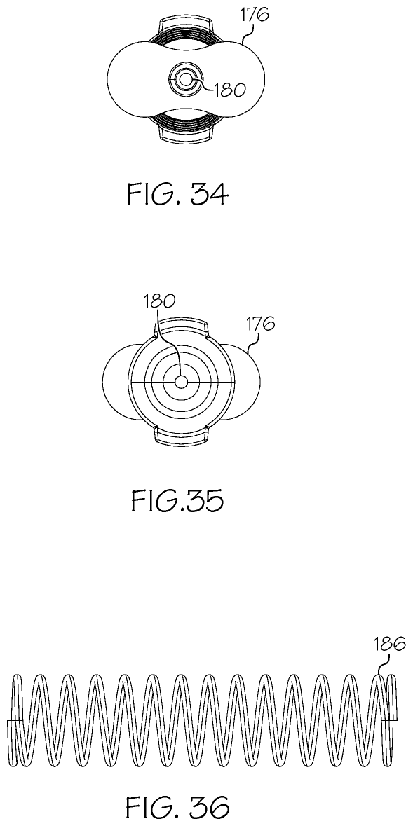

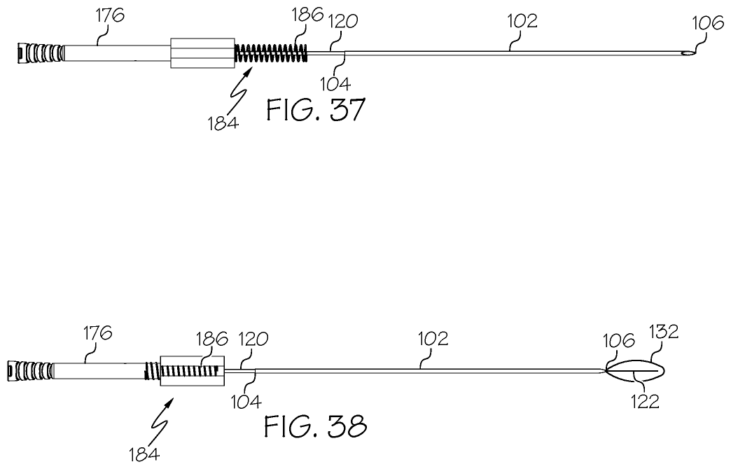

As shown in FIGS. 32-35, FIG. 37, and FIG. 38, in some embodiments the arc-and-loop suture grasper 100 further comprises a proximal hub 176 attached to the retriever body 120 and extending proximally therefrom. The proximal hub 176 is located at a proximal portion 178 of the arc-and-loop suture grasper 100. As shown in FIG. 33, the proximal hub 176 can have a proximal portion that functions as a button 190 for operating the arc-and-loop suture grasper 100. As shown in FIG. 34 and FIG. 35, the proximal hub 176 can have an open lumen 180. The proximal hub 176 can be Luer-lock compatible to allow connection of a syringe for administration or aspiration of fluids through the arc-and-loop suture grasper 100. In some embodiments, the proximal hub 176 is rigidly fixed to the retriever body 120. In some embodiments, the proximal hub 176 is rigidly fixed to the suture retrieval needle 102.

As shown in FIG. 1, in some embodiments the arc-and-loop suture grasper 100 further comprises a handle 182 attached to the suture retrieval needle 102. This is advantageous for improving the ergonomics of the arc-and-loop suture grasper 100 by allowing for single-handed operation. The handle 182 would preferentially include a non-axisymmetric design to rotationally fix the handle 182 and the suture retrieval needle 102.

The handle 182 can serve as a housing to contain internal components of the arc-and-loop suture grasper 100.

As shown in FIGS. 36-38, in some embodiments the arc-and-loop suture grasper 100 further comprises a return mechanism 184 configured to cause the arced grasper wire 122 to translate from the second position 130 to the first position 128 by translating the retriever body 120 proximally within the suture retrieval needle 102. The return mechanism 184 can comprise, for example, a spring 186. In these embodiments, the retriever body 120 can be attached to a proximal hub 176 having a proximal portion that functions as a button 190, directly or indirectly, so that when the button 190 is depressed, the spring 186 is compressed and the retriever body 120 is advanced, which in turn advances the arced grasper wire 122 and the looped grasper wire 132 from the needle lumen 116. When the button 190 is released, the spring 186 relaxes, returning the retriever body 120 to its initial position. This returns the arced grasper wire 122 and the looped grasper wire 132 to the needle lumen 116.

In some embodiments, the arc-and-loop suture grasper 100 further comprises a lock mechanism that can be reversibly engaged to prevent translation of the retriever body 120 within the needle lumen 116 in the first direction and/or the second direction. The lock mechanism can be advantageous for bearing fully a tensile load applied to a suture 300 in the same direction.

In some of these embodiments, the lock mechanism can be reversibly engaged in a first setting that prevents translation of the retriever body 120 within the needle lumen 116 in the first direction but not the second direction when the distal end 126 of the arced grasper wire 122 is inside the needle lumen 116 and reversibly engaged in a second setting that prevents translation of the retriever body 120 within the needle lumen 116 in the first direction but not the second direction when the distal-most loop point 140 of the grasper wire loop 138 is outside of the needle lumen 116. In some of these embodiments, maintaining the lock mechanism in the first setting or the second setting does not require energy input.

In some embodiments, the lock mechanism can be a reversible locking mechanism to maintain the arced grasper wire 122 in the first position 128. This can be advantageous for preventing the arced grasper wire 122 from being pulled distally from needle lumen 116 of the suture retrieval needle 102 if there is a significant tensile force on a suture 300 that is being grasped, and thus for preventing premature release of the suture 300.

Any suitable lock mechanism can be used with the arc-and-loop suture grasper 100. For example, the lock mechanism can include a simple lever arm molded into a proximal hub 176 of the arc-and-loop suture grasper 100 and serrations on the outer surface of a retriever body 120 such that the lever arm can engage with the serrations to lock the retriever body 120, and thus the arced grasper wire 122 and the looped grasper wire 132, against translation relative to the suture retrieval needle 102. The lock mechanism also can be a bolt-action lock mechanism in which a simple lever is moved within a J-shaped channel. An off-axis move into the short leg of the J-shaped channel secures the arm against the tensile force applied by a return spring. Another off-axis move repositions the lever into the long leg of the J-shaped channel to return to the original position. The lock mechanism also can be a side button lock mechanism in which a U-shaped flat spring floats within a moving member, and a spring action of the U-shaped flat spring causes the spring to engage with a lip in the moving member to lock the device in an extended position. The U-shaped spring protrudes from the side of the device, forming a button. Pressing the button disengages the U-shaped spring from the lip, allowing a return spring to return the device to a retracted position. The lock mechanism also can be a crossing-path lock mechanism, including a metal pin fixed in a floating carriage. The carriage is longitudinally fixed, but freely rotates, which allows the pin to follow grooves cut in a medial member. The grooves create a crossing path that locks and unlocks the device as the medial member is slidingly translated. The lock mechanism also can be a twist lock mechanism featuring a medial sleeve with a helical cut. When the sleeve is rotated, the helical surface acts upon a rotationally fixed cam follower to create linear motion. The lock mechanism also can be a detent mechanism. Other suitable lock mechanisms also can be used.

As noted above, in some embodiments the arc-and-loop suture grasper 100 further comprises a return mechanism 184 configured to cause the arced grasper wire 122 to translate from the second position 130 to the first position 128 by translating the retriever body 120 proximally within the suture retrieval needle 102. Also as noted, the return mechanism 184 can comprise, for example, a spring 186.

In some of these embodiments, the spring 186 has a spring constant, and the spring constant can be used to maintain the arced grasper wire 122 in the first position 128. This can be advantageous based on being easy and intuitive for surgeons to use and for less expensive manufacturing. This also can be advantageous by allowing the spring 186 to be tuned to physically release a suture 300 from the arc-and-loop suture grasper 100 at a predetermined tension lower than the tension at which one or more parts of the arc-and-loop suture grasper 100 would fail. This can be advantageous as a primary safety feature by decreasing the risk of a failure of individual parts of the arc-and-loop suture grasper 100, which might result in the one or more parts breaking and pieces thereof being dropped within a surgical field. Instead, the failure would simply involve the arc-and-loop suture grasper 100 dropping the suture based on the arced grasper wire 122 and the grasper wire loop 138 being pulled from needle lumen 116 as the spring 186 becomes increasingly compressed, resulting in release of the suture 300 from the arced grasper wire 122 and the grasper wire loop 138.

In some embodiments, the looped grasper wire 132 is made with a diameter sufficiently smaller relative to the diameter of the arced grasper wire 122 that, if excessive tension is applied on the arc-and-loop suture grasper 100 during use and the spring 186 does not compress sufficiently to result in the suture being dropped, then the looped grasper wire 132 would fail instead of the arced grasper wire 122. This can be advantageous as a secondary safety feature by decreasing the risk of the arced grasper wire 122 failing, which might result in a portion of the arced grasper wire 122 shearing and becoming lost within a patient. Instead, the looped grasper wire 132 would fail based on the grasper wire loop 138 shearing to form two legs, each remaining attached to the rest of the looped grasper wire 132. For a looped grasper wire 132 and an arced grasper wire 122 made from the shape memory alloy Nitinol we have determined that this can be accomplished by making the looped grasper wire 132 with a diameter of 0.005 inches (0.13 mm), and making the arced grasper wire 122 with a diameter of 0.008 inches (0.20 mm). Additional suitable combinations of diameters for the arced grasper wire 122 and the looped grasper wire 132 can be determined considering that ultimate tensile strength of a wire is directly proportional to the cross-sectional area of the wire and/or based on testing. For example, the arced grasper wire 122 can have a diameter of 0.0016 inches (0.040 mm) to 0.016 inches (0.40 mm), and the looped grasper wire 132 can have a diameter of 0.001 inches (0.025 mm) to 0.010 inches (0.25 mm), the diameter of the looped grasper wire 132 being smaller than the diameter of the arced grasper wire 122. Also for example, the arced grasper wire 122 can have a diameter of 0.008 inches (0.20 mm) to 0.016 inches (0.40 mm), and the looped grasper wire 132 can have a diameter of 0.003 inches (0.076 mm) to 0.007 inches (0.18 mm). Also for example, the arced grasper wire 122 can have a diameter of 0.006 inches (0.15 mm) to 0.009 inches (0.23 mm), and the looped grasper wire 132 can have a diameter of 0.003 inches (0.076 mm) to 0.005 inches (0.13 mm). Also for example, the arced grasper wire 122 can have a diameter of 0.0045 inches (0.11 mm) to 0.006 inches (0.15 mm), and the looped grasper wire 132 can have a diameter of 0.0025 inches (0.064 mm) to 0.004 inches (0.10 mm).

An exemplary arc-and-loop suture grasper 100 comprising a suture retrieval needle 102, a retriever body 120, an arced grasper wire 122, and a looped grasper wire 132, and further comprising a proximal hub 176 having a proximal portion having a button 190, a handle 182, and a return mechanism 184, as shown in FIGS. 1-18, can be operated through first and second stages of retraction as follows.

As shown in FIG. 45 and FIG. 46, with the button 190 depressed, the retriever tube 120 advanced, and the arced grasper wire 122 in the second position 130, and thus the arced grasper wire 122 and the grasper wire loop 138 extending from the suture retrieval needle 102, the arc-and-loop suture grasper 100 can manipulated to position the arced grasper wire 122 and the grasper wire loop 138 about a suture 300, such that the suture 300 is disposed between the arced grasper wire 122 and the grasper wire loop 138.

As shown in FIG. 47 and FIG. 48, during a first stage of retraction, when the button 190 is released, the stored energy in a spring 186 of the return mechanism 184 helps return the retriever tube 120 to its original position, which pulls the arced grasper wire 122 and the looped grasper wire 132 back inside the needle lumen 116 of the suture retrieval needle 102. The arced grasper wire 122 and the looped grasper wire 132 are crimped inside a retriever tube 150 of a retriever body 120 that can slide freely through the needle lumen 116 of the suture retrieval needle 102. The arced grasper wire 122 is designed such that a wall 192 of the suture retrieval needle 102 causes the arced grasper wire 122 to bend forward first, through the looped grasper wire 132. The mechanical energy of the spring 186 of the return mechanism 184 needs to be greater than the force required to elastically deform the arced grasper wire 122 and the looped grasper wire 132.

As shown in FIG. 49 and FIG. 50, during a second stage of retraction, after the arced grasper wire 122 and the looped grasper wire 132 have been fully retracted into the needle lumen 116 of the suture retrieval needle 102, the arced grasper wire 122 weaves through and past the looped grasper wire 132, and a suture 300 is being grasped by the arced grasper wire 122 and the looped grasper wire 132 within the needle lumen 116.

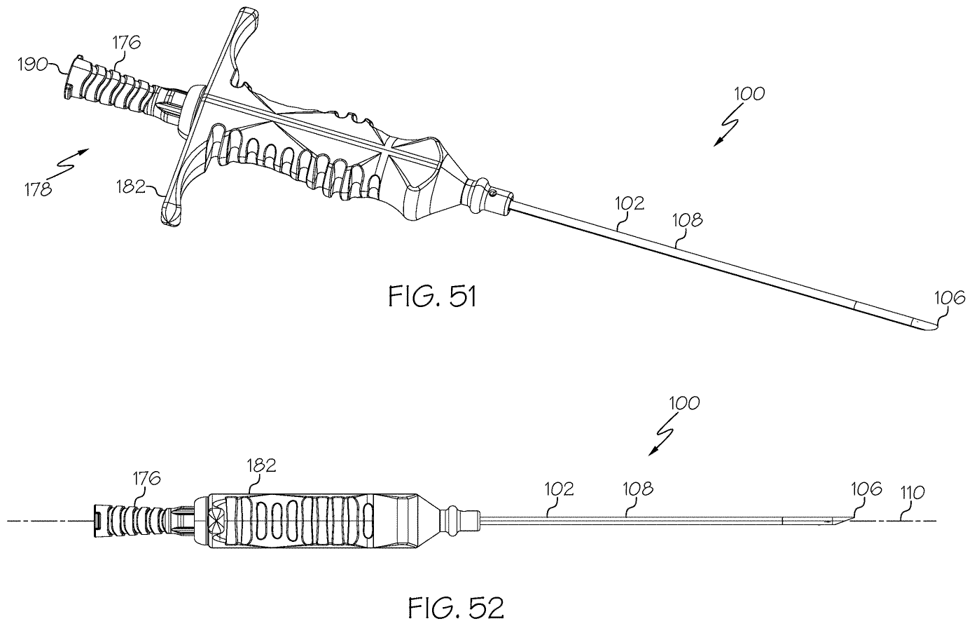

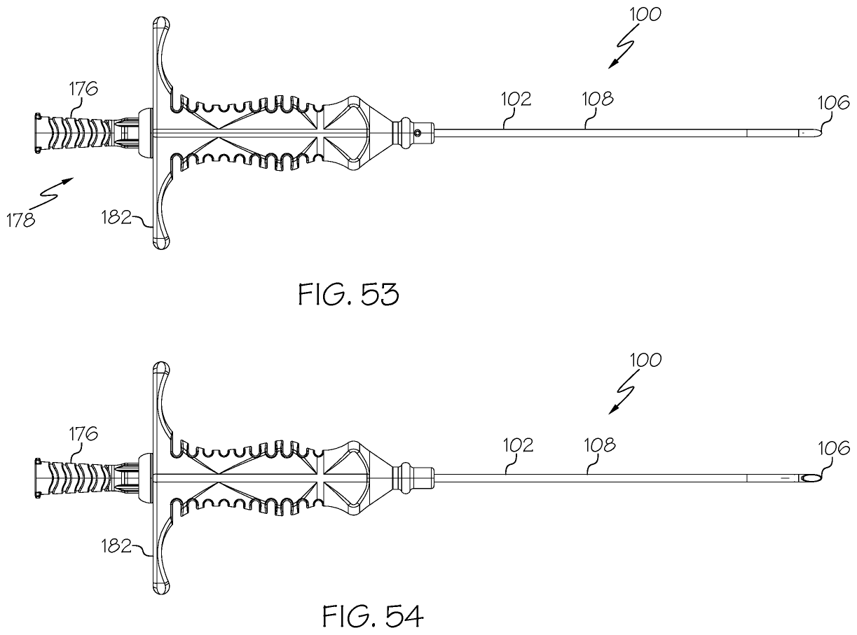

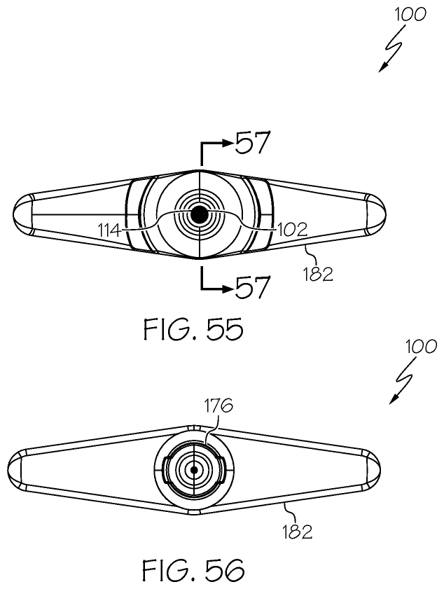

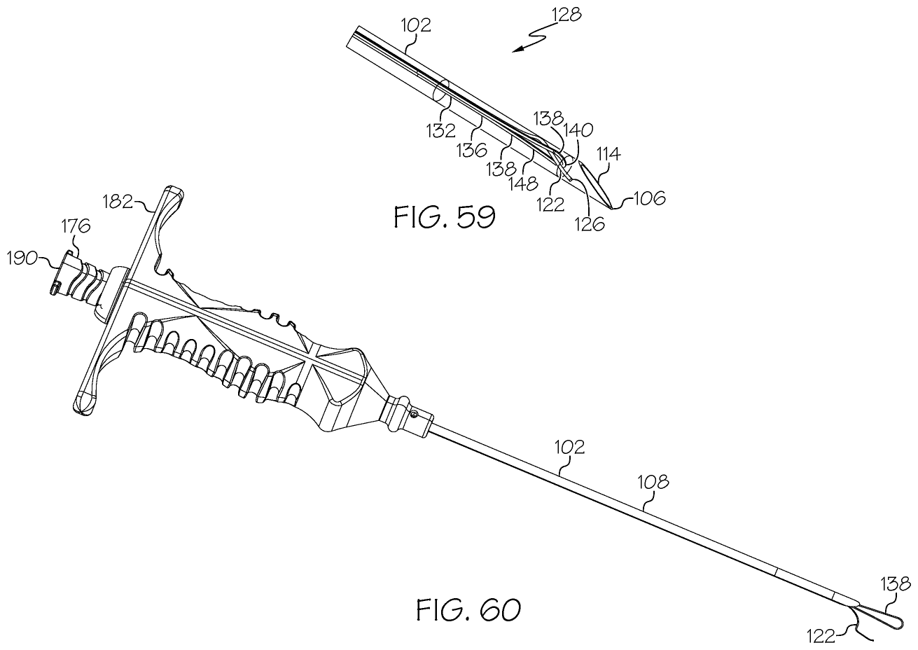

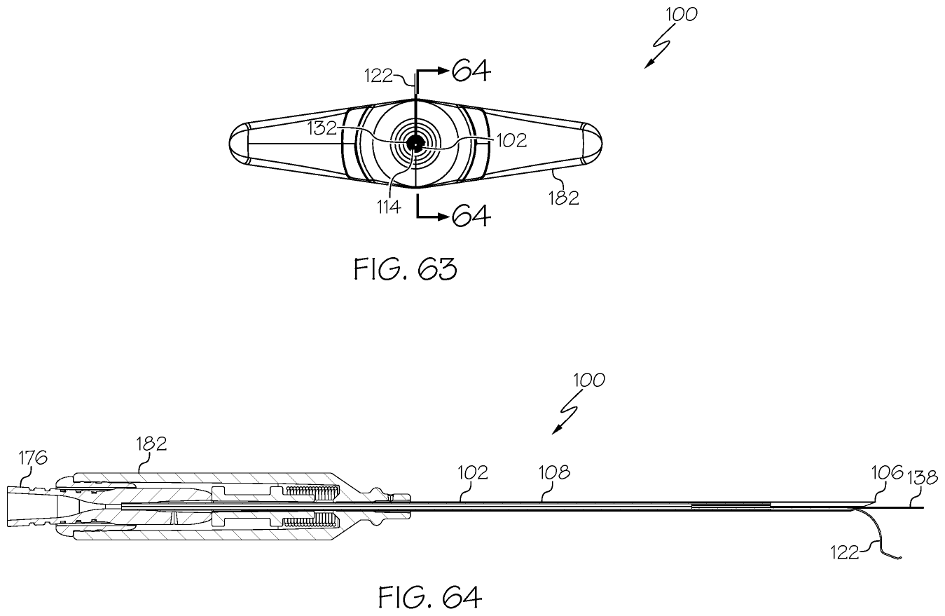

Another example embodiment of an arc-and-loop suture grasper 100 for grasping a suture 300 is shown in FIGS. 51-66, in which FIGS. 51-59 show the arc-and-loop suture grasper 100 with an arced grasper wire 122 in a first position 128, and the arc-and-loop suture grasper 100 sequestering a grasper wire loop 138 within a needle lumen 116, and FIGS. 60-66 show the arc-and-loop suture grasper 100 with the arced grasper wire 122 in the second position 130, and the arc-and-loop suture grasper 100 exposing the grasper wire loop 138 from the needle lumen 116.

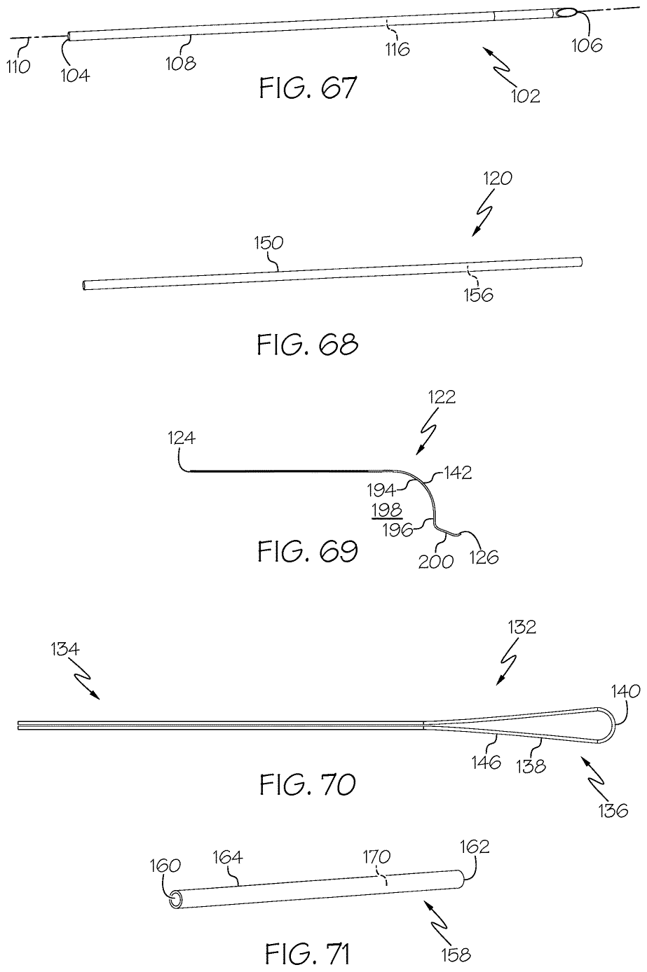

In accordance with this embodiment, as shown in FIGS. 67-70, the arc-and-loop suture grasper 100 comprises a suture retrieval needle 102, a retriever body 120, an arced grasper wire 122, and a looped grasper wire 132, all as described above.

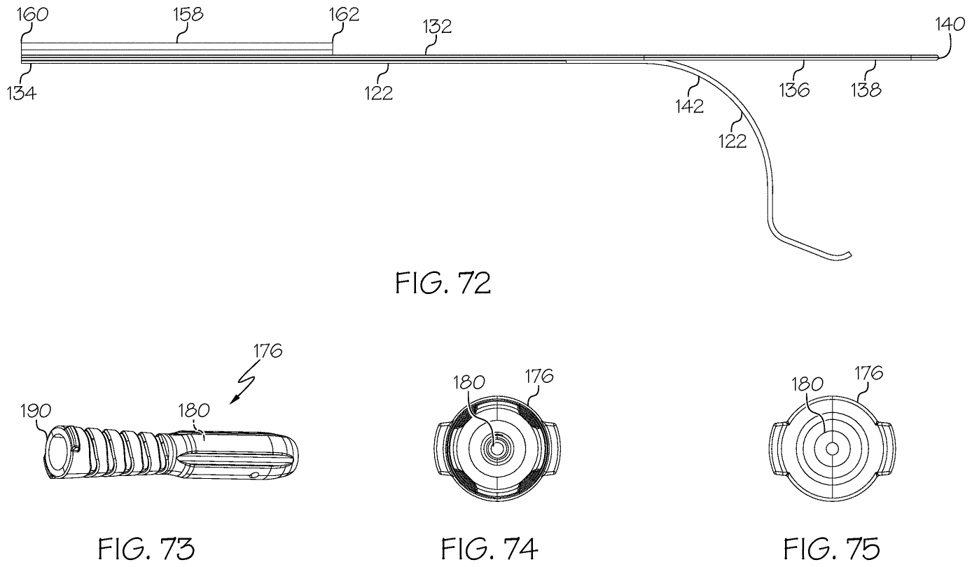

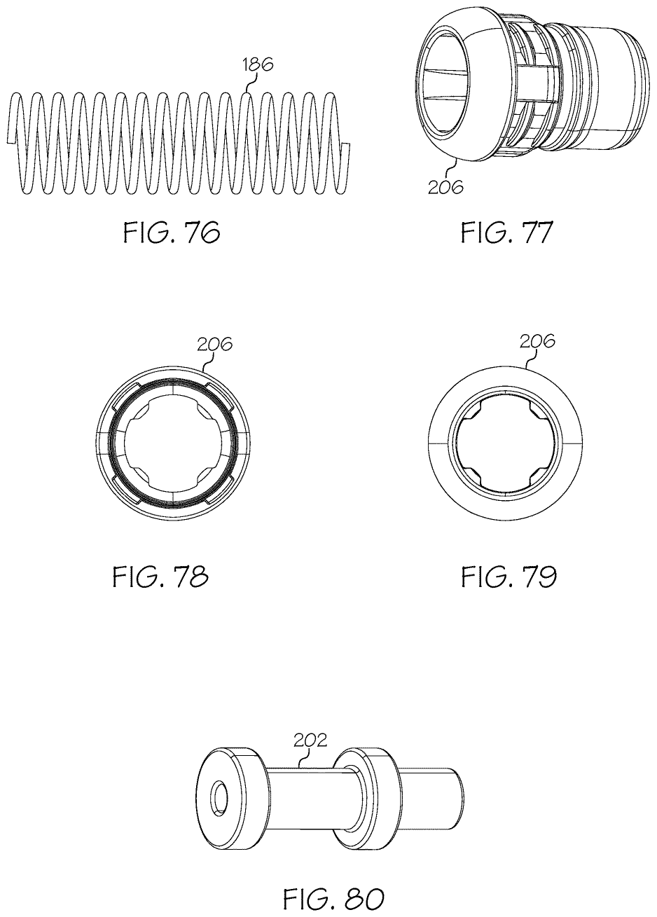

Also in accordance with this embodiment, as shown in FIGS. 71-76, with reference to FIG. 51 and FIG. 60, the arc-and-loop suture grasper 100 further comprises a grasper ferrule 158, a proximal hub 176 having a proximal portion that is a button 190, a handle 182, a return mechanism 184, and a spring 186, all also as described above.

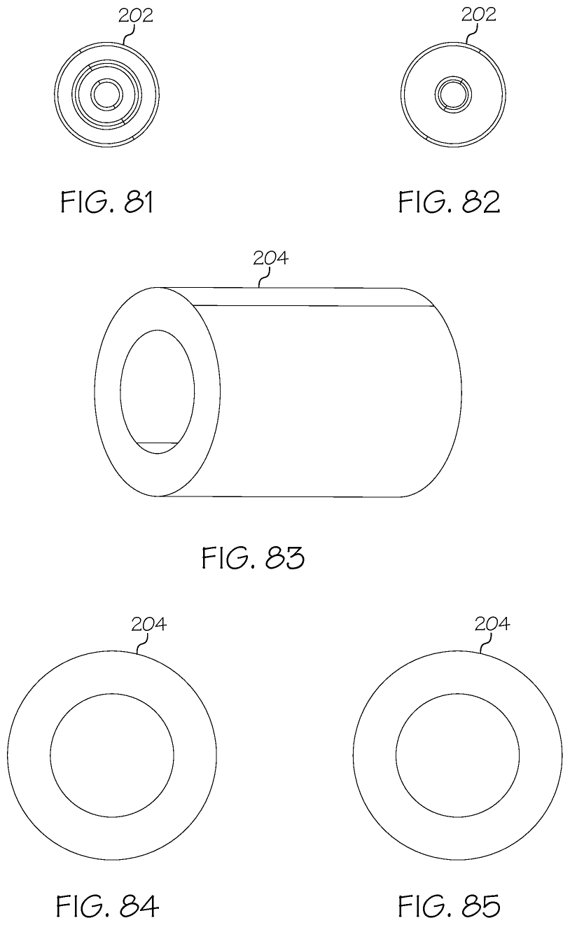

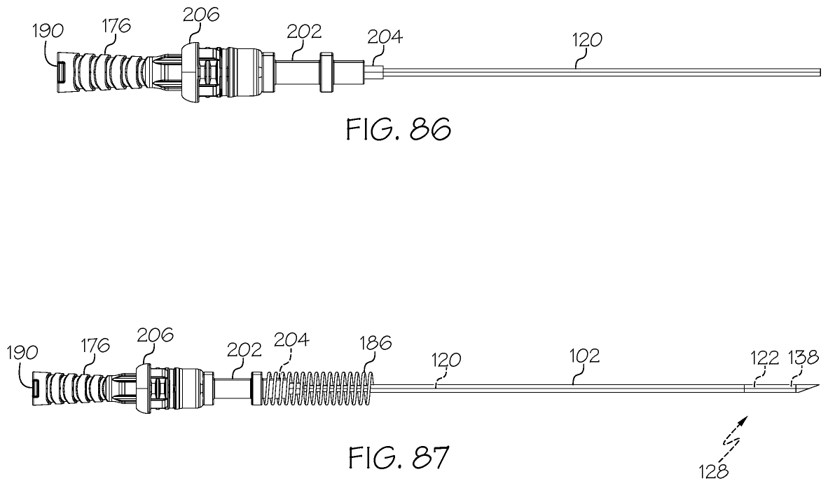

In accordance with this embodiment, as shown in FIGS. 77-88, the arc-and-loop suture grasper 100 further comprises a spring hat 202, a stay collar 204, and an end cap 206.

With reference to FIG. 87 and FIG. 88, the spring hat 202 can be a low-friction component that preloads the spring 186, maintaining tension, even when the arced grasper wire 122 is in the first position 128. The design of the spring hat 202 also determines available travel of the retriever body 120, proximal hub 176 having a proximal portion that is a button 190, the arced grasper wire 122, and the looped grasper wire 132 during use.

With reference to FIG. 87 and FIG. 88, the stay collar 204 is fixedly attached to the retriever body 120 and prevents the retriever body 120, the arced grasper wire 122, the looped grasper wire 132, and the spring hat 202 from over-traveling.

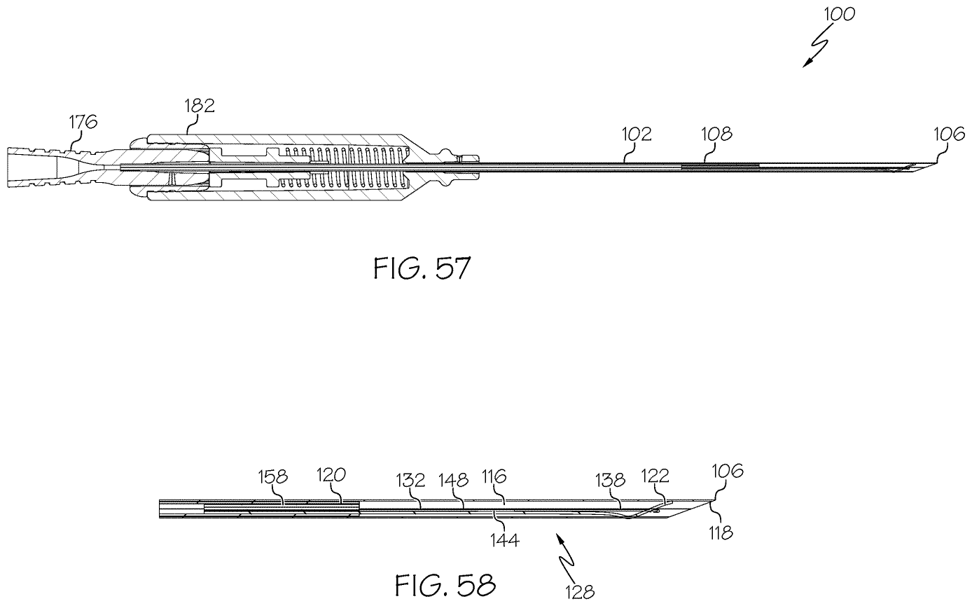

With reference to FIG. 57 and FIG. 64, the end cap 206 can be used to capture the retriever body 120, the spring hat 202, the spring 186, and the stay collar 204 within the handle 182.

The spring hat 202, the stay collar 204, and the end cap 206 work in tandem with the suture retrieval needle 102, the retriever body 120, the arced grasper wire 122, the looped grasper wire 132, the grasper ferrule 158, the proximal hub 176, the handle 182, the return mechanism 184, and the spring 186, further contributing to making the arc-and-loop suture grasper 100 a compact, secure, and ergonomic instrument tailored for surgeries.

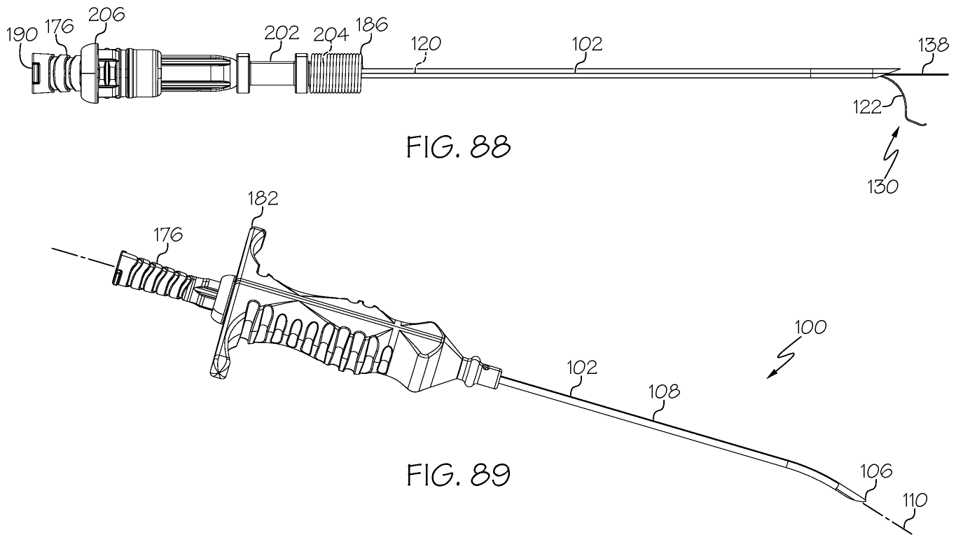

In accordance with this embodiment, as shown in FIG. 61, in some embodiments the suture retrieval needle 102 is straight. In accordance with these embodiments, the needle body axis 110 thereby is straight. Alternatively, as shown in FIG. 89, in some embodiments the suture retrieval needle 102 is curved. In accordance with these embodiments, the needle body axis 110 thereby is curved. As noted above, in some embodiments, one or more portions of the suture retrieval needle 102 can be straight, and one or more portions can be curved. For example, as shown in FIG. 89, in some embodiments the suture retrieval needle 102 includes a curve at or near its distal end 106 but otherwise is straight. In accordance with these embodiments, the needle body axis 110 also includes a curve at or near the distal end 106 of the suture retrieval needle 102, but otherwise is straight.

The arc-and-loop suture grasper 100 according to this embodiment can be operated as described above.

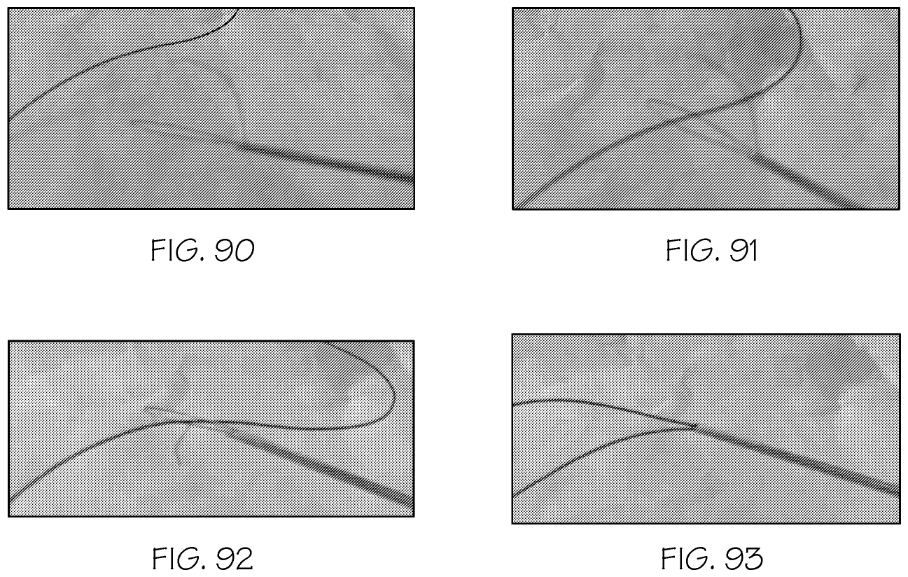

We have demonstrated that a prototype of our arc-and-loop suture grasper 100 can be used to capture and grasp a suture 300, as shown in FIGS. 90-93. FIG. 90 shows the arced grasper wire in the second position. A suture is also shown. FIG. 91 shows the suture fitting between the arced grasper wire and the grasper wire loop beyond the needle lumen. FIG. 92 shows the arced grasper wire in a position intermediate between the second position and the first position, and the suture disposed between the arced grasper wire and the grasper wire loop. FIG. 93 shows the arced grasper wire in the first position, the arced grasper wire having arced toward the grasper wire loop, the grasper wire loop sequestered, and the suture being grasped between the arced grasper wire and the grasper wire loop within the needle lumen.

The arc-and-loop suture grasper 100 disclosed herein may be useful in procedures including, among others: (1) inguinal hernia repair through high ligation of the patent processus vaginalis as described above; (2) laparoscopic port closure (typical suture size 2-0 and larger); (3) microsurgery (typical suture size 7-0 and smaller); (4) general surgery (typical suture size 2-0 to 0); and (5) orthopedic surgery (typical suture size 0 and larger).

A system 400 for passing a suture also is disclosed. The system 400 comprises the arc-and-loop suture grasper 100 and a suture 300. The arc-and-loop suture grasper 100 can be an arc-and-loop suture grasper 100 as described above. The suture 300 can be a conventional suture of any of a wide variety of suture types, styles, and sizes.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

### Clauses

1. An arc-and-loop suture grasper for grasping a suture comprising:
(a) a suture retrieval needle comprising a proximal end, a distal end, and a needle body extending therebetween, the needle body defining a needle body axis between the proximal and distal ends of the suture retrieval needle, the needle body having a proximal hole, a distal hole, and a needle lumen extending therebetween along the needle body axis;
(b) a retriever body disposed within the needle lumen and translatable therein along the needle body axis;
(c) an arced grasper wire having a proximal end and a distal end, the proximal end of the arced grasper wire being fixedly disposed within the retriever body, the arced grasper wire extending distally from the retriever body and being reversibly moveable between a first position and a second position; and
(d) a looped grasper wire comprising a proximal portion and a distal portion, the proximal portion of the looped grasper wire being fixedly disposed within the retriever body, the distal portion of the looped grasper wire forming a grasper wire loop, the grasper wire loop having a distal-most loop point, the looped grasper wire extending distally from the retriever body, the arc-and-loop suture grasper sequestering the grasper wire loop within the needle lumen when the arced grasper wire is in the first position and exposing the grasper wire loop from the needle lumen when the arced grasper wire is in the second position, wherein:
the arced grasper wire and the looped grasper wire are more flexible than the needle body;
when the arced grasper wire is in the first position the arced grasper wire extends through the grasper wire loop and the distal end of the arced grasper wire extends further distally than the distal-most loop point of the grasper wire loop;
when the arced grasper wire is in the second position the arced grasper wire arcs away from the grasper wire loop and the distal-most loop point of the grasper wire loop extends further distally than the distal end of the arced grasper wire;
the arced grasper wire is sufficiently short to exit the grasper wire loop as the arced grasper wire is moved from the first position to the second position and the looped grasper wire translates in unison with the arced grasper wire, and to enter the grasper wire loop as the arced grasper wire is moved from the second position to the first position and the looped grasper wire translates in unison with the arced grasper wire;
translation of the retriever body within the needle lumen in a first direction along the needle body axis causes the arced grasper wire to move from the first position to the second position, thereby allowing the arced grasper wire to exit the grasper wire loop and arc away from the grasper wire loop, exposing the grasper wire loop, and allowing a suture to fit between the arced grasper wire and the grasper wire loop beyond the needle lumen; and
translation of the retriever body within the needle lumen in a second direction opposite the first direction along the needle body axis causes the arced grasper wire to move from the second position to the first position, thereby causing the arced grasper wire to arc toward the grasper wire loop and enter the grasper wire loop, sequestering the grasper wire loop, and grasping the suture between the arced grasper wire and the grasper wire loop within the needle lumen.

2. The arc-and-loop suture grasper according to clause 1, wherein the suture retrieval needle is a hypodermic needle.

3. The arc-and-loop suture grasper according to clause 1 or clause 2, wherein the suture retrieval needle is straight, the needle body axis thereby being straight.

4. The arc-and-loop suture grasper according to clause 1 or clause 2, wherein the suture retrieval needle is curved, the needle body axis thereby being curved.

5. The arc-and-loop suture grasper according to any one of clauses 1 to 4, wherein the suture retrieval needle has a sharp tip.

6. The arc-and-loop suture grasper according to any one of clauses 1 to 5, wherein the retriever body comprises a retriever tube having a proximal hole, a distal hole, and a retriever tube lumen extending therebetween, the retriever tube being partially disposed within the needle lumen and translatable therein along the needle body axis, the proximal hole of the retriever tube being in fluid communication with the distal hole of the needle body through the retriever tube lumen and the needle lumen.

7. The arc-and-loop suture grasper according to clause 6, wherein the retriever tube comprises stainless steel.

8. The arc-and-loop suture grasper according to clause 6 or clause 7, wherein the retriever tube lumen is the only lumen of the retriever tube.

9. The arc-and-loop suture grasper according to any one of clauses 6 to 8, wherein translation of the retriever tube within the needle lumen is limited to a straight path as the translation of the retriever tube causes the arced grasper wire to move between the first and second positions.

10. The arc-and-loop suture grasper according to any one of clauses 6 to 9, further comprising a grasper ferrule comprising a proximal end, a distal end, and a ferrule body extending therebetween, the ferrule body having a proximal hole, a distal hole, and a ferrule lumen extending therebetween, the grasper ferrule being fixedly disposed within the retriever tube, wherein the arced grasper wire and the looped grasper wire extend distally from the grasper ferrule.

11. The arc-and-loop suture grasper according to clause 10, wherein:
the grasper ferrule has been crimped onto the arced grasper wire and the looped grasper wire with a crimped portion of the arced grasper wire and a crimped portion of the looped grasper wire being disposed within the ferrule lumen of the grasper ferrule; and
the retriever tube has been crimped onto the grasper ferrule, with the grasper ferrule, the crimped portion of the arced grasper wire, and the crimped portion of the looped grasper wire being disposed within the retriever tube lumen.

12. The arc-and-loop suture grasper according to clause 10, wherein the retriever tube has been crimped onto the grasper ferrule, the crimped portion of the arced grasper wire, and the crimped portion of the looped grasper wire, with the grasper ferrule, the crimped portion of the arced grasper wire, and the crimped portion of the looped grasper wire being disposed within the retriever tube lumen, and with the crimped portion of the arced grasper wire and the crimped portion of the looped grasper wire being disposed adjacent to the grasper ferrule and not within the ferrule lumen of the grasper ferrule.

13. The arc-and-loop suture grasper according to any one of clauses 1 to 12, wherein the arced grasper wire is formed from one or more of a shape memory alloy, titanium, stainless steel, or a monofilament polymer.

14. The arc-and-loop suture grasper according to any one of clauses 1 to 13, wherein the looped grasper wire is formed from one or more of a shape memory alloy, titanium, stainless steel, or a monofilament polymer.

15. The arc-and-loop suture grasper according to any one of clauses 1 to 14, wherein the arced grasper wire comprises a distal portion that forms an arcuate curve that defines an arcuate-curve interior between the grasper wire and the grasper wire loop when the arced grasper wire is in the second position.

16. The arc-and-loop suture grasper according to any one of clauses 1 to 14, wherein the arced grasper wire comprises a distal portion that forms an arcuate curve that defines an arcuate-curve interior not between the grasper wire and the grasper wire loop when the arced grasper wire is in the second position.

17. The arc-and-loop suture grasper according to any one of clauses 1 to 16, wherein the arced grasper wire comprises a distal-most portion that extends transversely from a distal portion of the arced grasper wire when the arced grasper wire is in the second position.

18. The arc-and-loop suture grasper according to any one of clauses 1 to 17, wherein the arced grasper wire and the looped grasper wire each have a diameter, and the diameter of the looped grasper wire is smaller than the diameter of the arced grasper wire.

19. The arc-and-loop suture grasper according to clause 18, wherein:
the arced grasper wire has a diameter of 0.0016 inches (0.040 mm) to 0.016 inches (0.40 mm), and the looped grasper wire has a diameter of 0.001 inches (0.025 mm) to 0.010 inches (0.25 mm);
the arced grasper wire has a diameter of 0.008 inches (0.20 mm) to 0.012 inches (0.30 mm), and the looped grasper wire has a diameter of 0.003 inches (0.076 mm) to 0.007 inches (0.18 mm);
the arced grasper wire has a diameter of 0.006 inches (0.15 mm) to 0.009 inches (0.23 mm), and the looped grasper wire has a diameter of 0.003 inches (0.076 mm) to 0.005 inches (0.13 mm); and/or
the arced grasper wire has a diameter of 0.0045 inches (0.11 mm) to 0.006 inches (0.15 mm), and the looped grasper wire has a diameter of 0.0025 inches (0.064 mm) to 0.004 inches (0.10 mm).

20. The arc-and-loop suture grasper according to any one of clauses 1 to 19, further comprising a proximal hub attached to the retriever body and extending proximally therefrom.

21. The arc-and-loop suture grasper according to clause 20, further comprising a handle attached to the retriever body.

22. The arc-and-loop suture grasper according to clause 21, further comprising a return mechanism configured to cause the arced grasper wire to translate from the second position to the first position by translating the retriever body proximally from within the suture retrieval needle, wherein the return mechanism comprises a spring.

23. The arc-and-loop suture grasper according to clause 22, wherein the spring has a spring constant, and the spring constant can be used to maintain the arced grasper wire in the first position.

24. The arc-and-loop suture grasper according to clause 23, further comprising a spring hat, a stay collar, and an end cap wherein:
the spring hat is a low-friction component that preloads the spring, maintaining tension, even when the arced grasper wire is in the first position, and that determines available travel of the retriever body, the proximal hub, the arced grasper wire, and the looped grasper wire during use;
the stay collar is fixedly attached to the retriever body and prevents the retriever body, the arced grasper wire, the looped grasper wire, and the spring hat from over-traveling; and
the end cap can be used to capture the retriever body, the spring hat, the spring, and the stay collar within the handle.

25. A system for passing a suture comprising:
the arc-and-loop suture grasper of any one of clauses 1 to 24; and
a suture.

What is claimed is:

1. An arc-and-loop suture grasper for grasping a suture comprising:
(a) a suture retrieval needle comprising a proximal end, a distal end, and a needle body extending therebetween, the needle body defining a needle body axis between the proximal and distal ends of the suture retrieval needle, the needle body having a proximal hole, a distal hole, and a needle lumen extending therebetween along the needle body axis;
(b) a retriever body disposed within the needle lumen and translatable therein along the needle body axis;
(c) an arced grasper wire having a proximal end and a distal end, the proximal end of the arced grasper wire being fixedly disposed within the retriever body, the arced grasper wire extending distally from the retriever body and being reversibly moveable between a first position and a second position; and
(d) a looped grasper wire comprising a proximal portion and a distal portion, the proximal portion of the looped grasper wire being fixedly disposed within the retriever body, the distal portion of the looped grasper wire forming a grasper wire loop, the grasper wire loop having a distal-most loop point, the looped grasper wire extending distally from the retriever body, the arc-and-loop suture grasper sequestering the grasper wire loop within the needle lumen when the arced grasper wire is in the first position and exposing the grasper wire loop from the needle lumen when the arced grasper wire is in the second position, wherein:
the arced grasper wire and the looped grasper wire are more flexible than the needle body;
when the arced grasper wire is in the first position the arced grasper wire extends through the grasper wire loop and the distal end of the arced grasper wire extends further distally than the distal-most loop point of the grasper wire loop;
when the arced grasper wire is in the second position the arced grasper wire arcs away from the grasper wire loop and the distal-most loop point of the grasper wire loop extends further distally than the distal end of the arced grasper wire;
the arced grasper wire is sufficiently short to exit the grasper wire loop as the arced grasper wire is moved from the first position to the second position and the looped grasper wire translates in unison with the arced grasper wire, and to enter the grasper wire loop as the arced grasper wire is moved from the second position to the first position and the looped grasper wire translates in unison with the arced grasper wire;
translation of the retriever body within the needle lumen in a first direction along the needle body axis causes the arced grasper wire to move from the first position to the second position, thereby allowing the arced grasper wire to exit the grasper wire loop and arc away from the grasper wire loop, exposing the grasper wire loop, and allowing a suture to fit between the arced grasper wire and the grasper wire loop beyond the needle lumen; and
translation of the retriever body within the needle lumen in a second direction opposite the first direction along the needle body axis causes the arced grasper wire to move from the second position to the first position, thereby causing the arced grasper wire to arc toward the grasper wire loop and enter the grasper wire loop, sequestering the grasper wire loop, and grasping the suture between the arced grasper wire and the grasper wire loop within the needle lumen.

2. The arc-and-loop suture grasper according to claim 1, wherein the suture retrieval needle is a hypodermic needle.

3. The arc-and-loop suture grasper according to claim 1, wherein the suture retrieval needle is straight, the needle body axis thereby being straight.

4. The arc-and-loop suture grasper according to claim 1, wherein the suture retrieval needle is curved, the needle body axis thereby being curved.

5. The arc-and-loop suture grasper according to claim 1, wherein the suture retrieval needle has a sharp tip.

6. The arc-and-loop suture grasper according to claim 1, wherein the retriever body comprises a retriever tube having a proximal hole, a distal hole, and a retriever tube lumen extending therebetween, the retriever tube being partially disposed within the needle lumen and translatable therein along the needle body axis, the proximal hole of the retriever tube being in fluid communication with the distal hole of the needle body through the retriever tube lumen and the needle lumen.

7. The arc-and-loop suture grasper according to claim 6, wherein the retriever tube comprises stainless steel.

8. The arc-and-loop suture grasper according to claim 6, wherein the retriever tube lumen is the only lumen of the retriever tube.

9. The arc-and-loop suture grasper according to claim 6, wherein translation of the retriever tube within the needle lumen is limited to a straight path as the translation of the retriever tube causes the arced grasper wire to move between the first and second positions.

10. The arc-and-loop suture grasper according to claim 6, further comprising a grasper ferrule comprising a proximal end, a distal end, and a ferrule body extending therebetween, the ferrule body having a proximal hole, a distal hole, and a ferrule lumen extending therebetween, the grasper ferrule being fixedly disposed within the retriever tube, wherein the arced grasper wire and the looped grasper wire extend distally from the grasper ferrule.

11. The arc-and-loop suture grasper according to claim 10, wherein:
the grasper ferrule has been crimped onto the arced grasper wire and the looped grasper wire with a crimped portion of the arced grasper wire and a crimped portion of the looped grasper wire being disposed within the ferrule lumen of the grasper ferrule; and
the retriever tube has been crimped onto the grasper ferrule, with the grasper ferrule, the crimped portion of the arced grasper wire, and the crimped portion of the looped grasper wire being disposed within the retriever tube lumen.

12. The arc-and-loop suture grasper according to claim 10, wherein the retriever tube has been crimped onto the grasper ferrule, the crimped portion of the arced grasper wire, and the crimped portion of the looped grasper wire, with the grasper ferrule, the crimped portion of the arced grasper wire, and the crimped portion of the looped grasper wire being disposed within the retriever tube lumen, and with the crimped portion of the arced grasper wire and the crimped portion of the looped grasper wire being disposed adjacent to the grasper ferrule and not within the ferrule lumen of the grasper ferrule.

13. The arc-and-loop suture grasper according to claim 1, wherein the arced grasper wire is formed from one or more of a shape memory alloy, titanium, stainless steel, or a monofilament polymer.

14. The arc-and-loop suture grasper according to claim 1, wherein the looped grasper wire is formed from one or more of a shape memory alloy, titanium, stainless steel, or a monofilament polymer.

15. The arc-and-loop suture grasper according to claim 1, wherein the arced grasper wire comprises a distal portion that forms an arcuate curve that defines an arcuate-curve interior between the grasper wire and the grasper wire loop when the arced grasper wire is in the second position.

16. The arc-and-loop suture grasper according to claim 1, wherein the arced grasper wire comprises a distal portion that forms an arcuate curve that defines an arcuate-curve interior not between the grasper wire and the grasper wire loop when the arced grasper wire is in the second position.

17. The arc-and-loop suture grasper according to claim 1, wherein the arced grasper wire comprises a distal-most portion that extends transversely from a distal portion of the arced grasper wire when the arced grasper wire is in the second position.

18. The arc-and-loop suture grasper according to claim 1, wherein the arced grasper wire and the looped grasper wire each have a diameter, and the diameter of the looped grasper wire is smaller than the diameter of the arced grasper wire.

19. The arc-and-loop suture grasper according to claim 18, wherein:
the arced grasper wire has a diameter of 0.0016 inches (0.040 mm) to 0.016 inches (0.40 mm), and the looped grasper wire has a diameter of 0.001 inches (0.025 mm) to 0.010 inches (0.25 mm);
the arced grasper wire has a diameter of 0.008 inches (0.20 mm) to 0.012 inches (0.30 mm), and the looped grasper wire has a diameter of 0.003 inches (0.076 mm) to 0.007 inches (0.18 mm);
the arced grasper wire has a diameter of 0.006 inches (0.15 mm) to 0.009 inches (0.23 mm), and the looped grasper wire has a diameter of 0.003 inches (0.076 mm) to 0.005 inches (0.13 mm); and/or
the arced grasper wire has a diameter of 0.0045 inches (0.11 mm) to 0.006 inches (0.15 mm), and the looped grasper wire has a diameter of 0.0025 inches (0.064 mm) to 0.004 inches (0.10 mm).

20. The arc-and-loop suture grasper according to claim 1, further comprising a proximal hub attached to the retriever body and extending proximally therefrom.

21. The arc-and-loop suture grasper according to claim 20, further comprising a handle attached to the retriever body.

22. The arc-and-loop suture grasper according to claim 21, further comprising a return mechanism configured to cause the arced grasper wire to translate from the second position to the first position by translating the retriever body proximally from within the suture retrieval needle, wherein the return mechanism comprises a spring.

23. The arc-and-loop suture grasper according to claim 22, wherein the spring has a spring constant, and the spring constant can be used to maintain the arced grasper wire in the first position.

24. The arc-and-loop suture grasper according to claim 23, further comprising a spring hat, a stay collar, and an end cap wherein:
the spring hat is a low-friction component that preloads the spring, maintaining tension, even when the arced grasper wire is in the first position, and that determines available travel of the retriever body, the proximal hub, the arced grasper wire, and the looped grasper wire during use;
the stay collar is fixedly attached to the retriever body and prevents the retriever body, the arced grasper wire, the looped grasper wire, and the spring hat from over-traveling; and
the end cap can be used to capture the retriever body, the spring hat, the spring, and the stay collar within the handle.

25. A system for passing a suture comprising:
the arc-and-loop suture grasper of claim 1; and
a suture.
